(19) United States
(12) Patent Application Publication
(10) Pub. No.: US 2024/0067910 A1
(43) Pub. Date: Feb. 29, 2024

(54) VISCOELASTIC MECHANOPORATION SYSTEMS AND METHODS OF USE THEREOF

(71) Applicant: The General Hospital Corporation, Boston, MA (US)

(72) Inventors: Derin Sevenler, Charlestown, MA (US); Mehmet Toner, Charlestown, MA (US)

(21) Appl. No.: 18/271,826

(22) PCT Filed: Jan. 12, 2022
(86) PCT No.: PCT/US2022/012098

§ 371 Date: Jul. 11, 2023

Related U.S. Application Data
(60) Provisional application No. 63/136,669, filed on Jan. 13, 2021.

(51) Int. Cl. C12M 3/06; C12M 1/42; C12N 5/00; C12N 15/113; C12N 15/87

(52) U.S. Cl. CPC C12M 23/16; C12M 35/04; C12N 5/0068; C12N 15/1138; C12N 15/87; C12N 2521/10

(57) ABSTRACT

Provided herein are methods of intracellular delivery of a substance to one or more cells. The methods include providing a substrate defining a micro-channel in fluid communication with a first chamber and optionally in fluid communication with a second chamber, the micro-channel having a hydraulic diameter that is less than a hydraulic diameter of the first and second chambers; and driving a cell suspension through the micro-channel, thereby: i) causing the one or more cells to be stretched along a direction of flow and ii) inducing a formation of one or more temporary pores in a membrane of the one or more cells, wherein the cell suspension comprises the one or more cells, a polymer, and the substance. Also provided are systems for the intracellular delivery of a substance to one or more cells.

### CROSS-REFERENCE TO RELATED APPLICATIONS

This application claims the benefit of U.S. Provisional Application No. 63/136,669, filed on Jan. 13, 2021. The entire contents of the foregoing are incorporated herein by reference in their entireties.

### FEDERALLY SPONSORED RESEARCH OR DEVELOPMENT

This invention was made with Government support under Grant Number R01CA255602 awarded by the National Cancer Institute of the National Institutes of Health. The Government has certain rights in the invention.

### TECHNICAL FIELD

This disclosure relates to systems and methods for intracellular delivery of a substance to one or more cells.

### BACKGROUND

Intracellular delivery of an effector molecule is an essential step in many biotechnology applications in medicine, biomanufacturing, and biological research. Historically, the most common effector biomolecule was a nucleic acid, but more recent applications increasingly require the delivery of other types of cargoes such as DNA-editing enzymes, complexes, and systems consisting of multiple biomolecular components or genes. For example, DNA and RNA transfection alone is no longer sufficient for precision gene editing with modern tools. In the case of the CRISPR/Cas9 system, the Cas9 endonuclease must bind with specific RNA(s) to form the functional ribonucleoprotein (RNP) complex. Importantly, forming the RNP in vitro before delivery avoids interference from endogenous RNA and increases editing efficiency. Delivering the complete functional RNP results in faster and more complete editing within 3-12 hours, and complete RNP degradation within 24 hours, whereas expression from a DNA plasmid can continue for at least 72 hours. This is important because CRISPR does not have perfect specificity and overexpression causes off-target DNA damage. DNA plasmids and viral vectors risk insertional mutagenesis, and DNA plasmids are stressful and damaging to many primary cell types including lymphocytes. mRNA delivery, especially with synthetic base analogs, can address some of these challenges but is still not as efficient as direct RNP delivery. As a result, direct delivery of RNPs formed in vitro is highly preferred for gene editing of human cells for therapeutic applications.

Traditional transfection technologies such as viral vectors, electroporation, and lipofection were developed for gene delivery are generally optimal for delivering nucleic acids to cell lines but have major limitations for delivering large and/or diverse biomolecular cargoes into primary mammalian cells. Viral vectors are limited to nucleic acid payloads and have significant limitations such as development time, difficult and laborious biomanufacturing, and limited payload capacity. Synthetic vectors are nonviral packaging systems that use polymers and lipids which bind to the cargo, protecting it from degradation and facilitating transport across the cell membrane. However, because they lack the specialized viral machinery for entering the cell, synthetic vectors are not efficient for immune cells, neurons, and stem cells.

In contrast to vectors, electroporation is a more capable and flexible system for delivering different cargos to multiple cell types because it directly permeabilizes the plasma membrane of any mammalian cell; however, electroporation has major caveats. First, delivery is highly dependent on the size and mass charge ratio of the cargo. Neutrally charged biomolecules such as proteins do not benefit from the electrophoretic force. Delivering large and/or uncharged biomolecules also require high voltage and long pulses to create large pores and keep them open. As a result, large swaths of membrane including intracellular membranes are permeabilized, leading to mitochondrial damage, ion and pH imbalance, calcium influx, ATP depletion, and necrosis. Third, electroporation is fundamentally limited in its ability to deliver large cargos. Increasing voltage has diminishing returns on pore size above 25 nm due to pore conductivity.

Groundbreaking new cell and gene therapies are revolutionizing oncology and other medical fields but face unprecedented challenges in biomanufacturing. For example, dosages of chimeric antigen receptor (CAR) T therapy are usually about 1 to 10 million cells per kilogram, requiring up to 500 million engineered T cells per dose. Gene therapies using adeno-associated viral vector use up to 3×10¹⁴ viral genomes per kg, or roughly 10¹⁶ vg per dose. Production of this quantity of virus using mammalian cell factories is an extraordinary feat, involving over 500 HYPERstack-36™ systems—each containing 240× the culture area of a typical T75 flask—and altogether about 1.5 trillion virus-producing cells. As a result, for transfection methods to be relevant to clinical-scale biomanufacturing, their production throughput must be measured in units of billions to trillions of cells. No vector-free methods exist to deliver DNA-editing enzymes into cells at this scale. Osmotic stress, heat, cavitation, friction, and fluid forces have been explored as methods of disrupting the cell membrane to deliver materials inside; however, they have failed to address the field's needs because they have major practical limitations.

The most important limitation is that none of them are anywhere near fast or reliable enough to transfect billions of primary cells. The fastest so-called “high throughput” methods are still about 10× slower than microfluidic electroporation, which is itself perhaps 100× too slow for clinical-scale biomanufacturing and has fundamental problems described above. Also, many methods require physical contact with a surface or microscale tip, which inevitably leads to fouling and/or clogging. Non-contact methods using ultrasound (‘sonoporation’) are also extremely slow while remaining inefficient and nonuniform. Recently, hydraulic forces (‘hydroporation’) have been used to stretch and permeabilize cells without touching a solid surface, but this approach is impractical because it requires precisely positioning cells at a flow singularity, saddle point, or stagnation point in a very dilute solution. So-called ‘biolistic’ methods using nano-bullets to pierce cells are useful for in situ transfections but have fallen out of favor because they are generally much less efficient (often less than 10%) while remaining at least as toxic as electroporation. Altogether, existing methods including “high throughput” electroporation are still 100× to 1,000× too slow for clinical applications. More broadly, existing methods are not amenable to clinical samples or automation because the devices are complex (i.e., not disposable), prone to fouling, and require unfeasible amounts of cargo material.

Gene editing systems such as CRISPR hold enormous promise for cell and gene therapy, but clinical applications require billions of cells and current methods of delivering CRISPR are not nearly fast enough. There is an urgent unmet need for a method that can quickly and efficiently deliver DNA-editing enzymes into billions of cells. More broadly, there is a need for a technique for gentle, vector-free transfection of proteins and nanomaterials that may enable control of the molecular information inside of cells.

### SUMMARY

This disclosure relates to the viscoelastic mechanoporation of cells using micro-channels. The techniques and systems described herein can be implemented to achieve high throughput intracellular delivery of one or more substances to one or more cells (e.g., up to a billion cells per minute) by stretching the cells suspended in a viscoelastic solution until temporary pores are formed, and the substance is delivered through the temporary pores.

Certain aspects of the present disclosure are directed methods of intracellular delivery of a substance to one or more cells. The methods include providing a substrate in fluid communication with a first chamber and optionally in fluid communication with a second chamber. The micro-channel has a hydraulic diameter that is less than a hydraulic diameter of the first and second chambers. The methods include driving a cell suspension through the micro-channel, thereby: i) causing the one or more cells to be stretched along a direction of flow and ii) inducing a formation of one or more temporary pores in a membrane of the one or more cells, wherein the cell suspension comprises the one or more cells, a polymer, and the substance.

In some embodiments, the substance has a molecular mass ranging from about 0.1 megaDaltons (MDa) to about 2 MDa. In some embodiments, the substance comprises a protein, a nucleic acid, a nanomaterial, a drug, a catalyst, a polysaccharide, a bacterium, an organelle, a contrast agent, a molecular probe, a dye, or any combination thereof. In some embodiments, the substance has a diameter ranging from about 1 nm to about 100 nm. In some embodiments, the one or more cells are present in the cell suspension at a concentration ranging from about 1 million cells/ml to about 200 million cells/ml. In some embodiments, the one or more cells are stretched the most at a circumferential region around or near a center of each of the one or more cells.

In some embodiments, the driving of the cell suspension through the micro-channel causes an advection of the cell suspension toward the circumferential region. In some embodiments, the advection causes the substance to be delivered through the one or more temporary pores and into a cytosol of the one or more cells. In some embodiments, the one or more cells do not contact a surface of the micro-channel or of another cell of the one or more cells. In some embodiments, the driving of the cell suspension through the micro-channel creates a viscoelastic extensional flow. In some embodiments, the viscoelastic extensional flow is a uniaxial extensional flow, a biaxial extensional flow, or a combination thereof. In some embodiments, the cell suspension is driven through the micro-channel at a volumetric flow rate ranging from about 0.1 ml/minute to about 20 ml/minute. In some embodiments, the cell suspension is driven through the micro-channel at a volumetric flow rate resulting in a Weissenberg number ranging from about 1 to about 100. In some embodiments, the cell suspension is driven through the micro-channel by applying a driving pressure ranging from about 20 millibar (mbar) to about 7 bar.

In some embodiments, the driving pressure ranges from about 1 bar to about 3 bar. In some embodiments, the cell suspension has one or more viscoelastic properties. In some embodiments, the cell suspension is not a Newtonian fluid. In some embodiments, the cell suspension is accelerated while it is driven through the micro-channel. In some embodiments, the cell suspension comprises calcium ions. In some embodiments, the cell suspension comprises calcium ions at a concentration of about 1 μM to about 5 mM. In some embodiments, the one or more temporary pores have a diameter ranging from about 5 nm to about 100 nm. In some embodiments, the one or more temporary pores are sealed by the one or more cells within about 20 seconds after their formation. In some embodiments, the polymer comprises hyaluronic acid.

In some embodiments, the polymer comprises poly(ethylene) glycol, poly(vinyl) pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate polycaprolactone, cellulose, lignin, alginate, chitosan, starch, or any combination thereof.

In some embodiments, the polymer is present at a concentration ranging from about 0.001 mg/ml to about 4 mg/ml. In some embodiments, the polymer is present at a concentration ranging from about 0.1 mg/ml to about 3 mg/ml. In some embodiments, the polymer is present at a concentration ranging from about 2 mg/ml to about 4 mg/ml. In some embodiments, the methods further include focusing the one or more cells into a localized fluid flow path, wherein the one or more cells are located within a same distance from each other.

In another aspect, the present disclosure is directed to methods of delivery of a substance to one or more particles, the method comprising: providing a substrate defining a micro-channel that connects a first chamber and a second chamber, the micro-channel having a hydraulic diameter that is less than a hydraulic diameter of the first and second chambers; and driving a particle suspension through the micro-channel, thereby: i) causing the one or more particles to be stretched along a direction of flow and ii) inducing a formation of one or more temporary pores in a membrane of the one or more particles, wherein the particle suspension comprises the one or more particles, a polymer, and the substance.

In some embodiments, the one or more particles are one or more liposomes, vesicles, exosomes, or any combination thereof.

In another aspect, the present disclosure is directed to microfluidic systems for intracellular delivery of a substance to one or more cells, the microfluidic system comprising: a substrate defining a micro-channel that connects a first chamber and optionally a second chamber, the micro-channel having a hydraulic diameter that is less than a hydraulic diameter of the first and second chambers; a cell suspension comprising the one or more cells, a polymer, and the substance; a pump configured to drive the cell suspension through the micro-channel, thereby: i) causing the one or more cells to be stretched along a direction of flow and ii) inducing a formation of one or more temporary pores in a membrane of the one or more cells; and optionally comprising a controller configured to control operation of the pump.

In some embodiments, the hydraulic diameter of the micro-channel is greater than a diameter of the one or more cells. In some embodiments, the diameter of the one or more cells ranges from about 4 nm to about 25 nm. In some embodiments, the hydraulic diameter of the micro-channel ranges from about 30 μm to about 100 μm. In some embodiments, the hydraulic diameter of the micro-channel is about 30 μm. In some embodiments, a length of the micro-channel is about 10 μm to about 500 μm. In some embodiments, the length of the micro-channel is about 100 μm. In some embodiments, the first and second chambers are tapered such that the hydraulic diameter of the first and second chambers gradually narrows prior to intersecting an opening of the micro-channel.

In some embodiments, a ratio of the cross-sectional area of the first or second chambers to the cross-sectional area of the micro-channel ranges from about 10 to about 500. In some embodiments, the ratio is about 150. In some embodiments, the hydraulic diameter of the first and second chambers ranges from about 1,000 μm to about 10,000 μm. In some embodiments, the hydraulic diameter of the first and second chambers is about 1,500 μm. In some embodiments, the pump is configured to drive the cell suspension from the first chamber, through the micro-channel, and to the second chamber. In some embodiments, the controller is configured to control the pump to drive the cell suspension through the micro-channel at a volumetric flow rate ranging from about 0.1 ml/minute to about 20 ml/minute.

In some embodiments, the controller is configured to control the pump to drive the cell suspension through the micro-channel at a volumetric flow rate resulting in a maximum Reynolds number of a flow ranging from about 10 to about 100. In some embodiments, the micro-channel is a first micro-channel and the system further comprises a second or more micro-channels connecting the first or second chambers to a third or more chambers in parallel. In some embodiments, the systems further include about 10 micro-channels to about 20 micro-channels. In some embodiments, the pump is configured to drive the cell suspension through the micro-channel by applying a driving pressure. In some embodiments, the system further includes a valve in fluid communication with the micro-channel and a pressure line, the valve configured to selectively control the driving pressure.

A “sample” (sometimes referred to as “fluid” or “fluid sample”) is capable of flowing through the micro-channel. The sample can include one or more of a fluid suspension or any sample that can be put into the form of a fluid suspension, and that can be driven through the micro-channel.

A “fluid” can include any type of fluid, e.g., water, a buffer, a cell culture medium. The particles dispersed in the fluid can include biological particles, e.g., mammalian cells (e.g., human cells) such as immune cells (e.g., T cells), stem cells, tumor cells, red blood cells, white blood cells, non-mammalian cells, or other types of biological particles that occur either naturally or are introduced artificially into the fluid. The particles dispersed in the fluid can also include non-biological or synthetic particles, e.g., lipid nanoparticles or lipid vesicles.

The term “substance” that is delivered using the methods and systems of the disclosure, as used herein, refers to a cargo, payload, agent, or material that can be dispersed in the delivery suspension. For example, the “substance” that is delivered using the methods and systems of the disclosure includes, but is not limited to, proteins, nanomaterials, nucleic acids, a drug, a catalyst, a polysaccharide, a bacterium, an organelle, a contrast agent, a molecular probe, a dye, or any combination thereof.

Particles (e.g., cells) suspended within a sample can have any size which allows them to be stretched within the microfluidic channel. For example, particles can have a hydrodynamic size that is between 1 μm and 100 μm. The particle size is limited only by micro-channel geometry; accordingly, particles that are larger and smaller than the above-described particles and can be transported through the micro-channel can be used. In some embodiments, the micro-channel minimum width can be fabricated to be larger to permit delivery to larger particles or fabricated smaller to enable delivery to smaller particles.

In some implementations, focusing (sometimes referred to as “localizing”) can be achieved by varying a flow rate of a fluid carrying suspended particles flowed through a channel of constant cross-section. In some implementations, focusing can be achieved by a reduction in the area of a cross-section of a channel through which a flux of particles passes. Particles can be localized within an area having a hydraulic diameter of, e.g., 0.5, 1, 2, 3, 4, or 5 times the diameter of the particles. Localization can occur at any location within the channel, e.g., at an unobstructed portion of the channel. Localization can occur in a portion of the channel having less than 50%, 40%, 30%, 20%, 10%, 5%, 2%, 1%, or 0.1% reduction in cross-sectional area.

By the term “nanoparticle” is meant an object that has a diameter between about 2 nm to about 200 nm (e.g., between 10 nm and 200 nm, between 2 nm and 100 nm, between 2 nm and 40 nm, between 2 nm and 30 nm, between 2 nm and 20 nm, between 2 nm and 15 nm, between 100 nm and 200 nm, and between 150 nm and 200 nm). Non-limiting examples of nanoparticles include the nanoparticles described herein.

By the term “nucleic acid” is meant any single- or double-stranded polynucleotide (e.g., DNA or RNA having a semi-synthetic or a synthetic origin). The term nucleic acid includes oligonucleotides containing at least one modified nucleotide (e.g., containing a modification in the base and/or a modification in the sugar) and/or a modification in the phosphodiester bond linking two nucleotides. Non-limiting examples of nucleic acids are described herein. Additional examples of nucleic acids are known in the art.

As used in the specification and the appended claims, the singular forms “a,” “an” and “the” include plural referents unless the context clearly dictates otherwise. Thus, for example, reference to “a nanoparticle” includes mixtures of nanoparticles, reference to “a nanoparticle” includes mixtures of two or more such nanoparticles, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Where values are described in the present disclosure in terms of ranges, endpoints are included. Furthermore, it should be understood that the description includes the disclosure of all possible sub-ranges within such ranges, as well as specific numerical values that fall within such ranges irrespective of whether a specific numerical value or specific sub-range is expressly stated.

Other features and advantages of the present disclosure will be apparent from the following detailed description and figures, and from the claims.

Various embodiments of the features of this disclosure are described herein. However, it should be understood that such embodiments are provided merely by way of example, and numerous variations, changes, and substitutions can occur according to those skilled in the art without departing from the scope of this disclosure. It should also be understood that various alternatives to the specific embodiments described herein are also within the scope of this disclosure.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features and advantages of the invention will be apparent from the following detailed description and figures, and from the claims.

### DESCRIPTION OF DRAWINGS

FIGS. 1A and 1B are schematics (not to scale) showing the intracellular delivery of a substance using an exemplary viscoelastic cell stretching method and system of the disclosure.

FIG. 2 is a schematic showing an exemplary overview of viscoelastic mechanoporation. Geometry, relative cell size, and cell deformation are approximately to scale.

FIG. 3 is a schematic showing cell behavior in a Newtonian solution and a viscoelastic solution. Shear stress, which emerges from the gradient in the flow velocity due to wall drag, results in uneven mechanoporation for cells at different distances from the wall. In a viscoelastic solution under extensional flow, normal stress differences arise across all streamlines, and are generally much larger than shear stresses. As a result, all cells are stretched without rotation.

FIGS. 4A-4D are microscopy images of an exemplary microfluidic geometry. A cross-section of the channel is shown in FIG. 4D. A single microfluidic channel connects two tapered chambers approximately 125 microns in height and 1,500 microns in width. The channel dimensions can be varied. The channel length can be between 0 microns and 400 microns long, and has cross section dimensions which can vary between 30 microns and 100 microns.

FIG. 5 is an image of an exemplary microfluidic system. 10 μL to 100 μL of delivery suspension, containing the cells, the cargo molecule, and the polymer (e.g., hyaluronic acid at a concentration of about 2 mg/ml), is loaded into a length of tubing which is connected to the microfluidic chip. A valve is turned, applying regulated pressure and driving the flow. The sample is fed through the outlet tubing to a collection tube.

FIG. 6A is a side, cross-sectional view of an example three-dimensional printed, microfluidic cell stretching device that is compatible with microwell plates. FIG. 6B is a side, perspective view of the device of FIG. 6A. FIG. 6C is a top, perspective view of the device of FIG. 6A.

FIG. 7A shows microscopy images of Jurkat cell deformation in 0.5% poly(ethylene oxide) solution at increasing flow rates. Flow is left to right. FIG. 7B shows representative images of segmentation by convolutional neural network.

FIGS. 8A and 8B are graphs showing quantification of Jurkat cell deformation in viscoelastic extensional flow at 0.05 bar and at 2.5 bar, respectively. Fluid flow is left to right.

FIGS. 9A and 9B are graphs showing the delivery efficiency of 2,000 kDa FITC-dextran to Jurkat T cells at increasing operating pressures in a delivery suspension containing (FIG. 9A) 1 mg/mL hyaluronic acid, or (FIG. 9B) no polymer.

FIGS. 10A-10C are graphs showing the delivery, viability, and viable cell recovery, respectively, for Jurkat cells at increasing pressures, using a 100 μm long channel with 45 μm by 60 μm cross-section.

FIG. 11 is a graph showing the delivery efficiency and cell viability of stretched Jurkat cells relative to unstretched Jurkat cells 24 hours after stretching (N=3) at various pressures.

FIG. 12A is a graph showing the delivery efficiency of FITC-Dextran 2000 kDa and cell viability. FIG. 12B is a graph showing the normalized cell yield at increasing cell concentrations up to 1×10⁸ cells per ml.

FIGS. 13A and 13B are graphs showing the delivery efficiency and cell viability, respectively, as a function of applied pressure. FIG. 13C shows histograms of delivery efficiency as a function of applied pressure for two exemplary channel geometries: the “zero-length channel” (shown on the left) and the “100 μm-channel” (shown on the right). The length of the channel affects the delivery efficiency of 2,000 kDa FITC-Dextran as well as cell viability. The “zero-length channel” (shown on the left) has higher delivery efficiency than the but at the expense of cell viability. Furthermore, the addition of a channel at least 100 microns long results in a clear bimodal distribution, suggesting efficient delivery to all permeabilized cells.

FIGS. 14A-14C are graphs showing a biomolecule cargo delivery is completed within seconds. FIG. 14D is a graph showing the amount of 2,000 kDa FITC-Dextran delivered is greatly reduced if added to the delivery suspension 5 seconds after stretching. Immediate dilution of the sample (within 5 seconds) in cargo-free culture medium has no effect on delivery.

FIG. 15 is a graph showing the delivery of 2,000 kDa dextran to T cells, 2 hours after stretching (N=1).

FIG. 16 is a graph showing the delivery of 70 kDa Dextran to the acute myeloid leukemia cell line MOLM-13, 2 hours after stretching (N=1).

FIG. 17A is a schematic showing an exemplary method of the delivery of Atto550 labeled CRISPR/Cas9 ribonucleoprotein complex. The crRNA is designed to target the TRAC locus of the T cell receptor. The tracRNA is tagged with the fluorophore Atto550. FIGS. 17B-17D are graphs showing the fluorescence intensity of the atto550 signal after 90 minutes in non-transfected cells, transfected and unstretched cells, and transfected and stretched cells, respectively. Knockout of the T cell receptor was assessed after 2 days of culture by immunofluorescence. FIGS. 17E-17G are graphs showing the fluorescence intensity of the T cell receptor in non-transfected cells, transfected and unstretched cells, and transfected and stretched cells, respectively. Delivery of Cas9/sgRNA ribonucleoprotein complex (RNP) by cell stretching resulted in about 70% delivery efficiency of RNP by atto550 signal, and about 60% relative reduction in T cell receptor expression by immunofluorescence.

FIG. 18A is a graph showing the delivery efficiency of RNP to Jurkat cells by atto550 fluorescence. FIG. 18B is a graph showing the T cell receptor expression 2 days after RNP delivery (N=2). *:p<0.05, **:p<0.01 by two-tailed t-test.

FIG. 19 is a graph showing finite element modeling of cell deformation and plasma membrane areal strain (light gray is high) in response to viscoelastic extensional flow.

FIG. 20 is a schematic illustrating particles streamlined into an exemplary localized fluid flow path.

FIG. 21A is a schematic illustrating a device including a channel having a spiral geometry. FIG. 21B is a schematic illustrating a device including two particle-focusing channels having a spiral geometry, which converge to a single constriction.

### DETAILED DESCRIPTION

The methods described herein are ultra-high throughput microfluidic methods of physically stretching the plasma membrane of cells to create temporary pores and facilitate efficient delivery of nanoscale cargoes. Viscoelastic fluid forces that emerge during flow acceleration are used to physically focus and stretch the cells using the micro-channels of the disclosure. Thus, cells in the delivery suspension are stretched without rotation anywhere in the flow and without contacting any surface. Micro-channel systems for intracellular delivery of substances to cells are also provided herein. Some embodiments of the therapeutic nanoparticles, compositions, and methods described herein may provide one or more of the following advantages.

First, certain embodiments of the present disclosure include methods of intracellular delivery of a substance or cargo to one or more cells. As discussed above, there is currently an unmet need for a high throughput method that can quickly and efficiently deliver DNA-editing enzymes into billions of cells. The methods and systems of the present disclosure address this need. For example, in some embodiments, the methods and systems described herein can achieve intracellular delivery of a cargo at a rate of over 50 million cells per minute with a single micro-channel. In some embodiments, the methods and systems described herein can achieve a throughput of 1 billion cells per minute with a system that includes only about 10 to 20 microchannels. Thus, in some embodiments, a unique property of the methods and systems described herein is its fast throughput speed compared to existing and emerging transfection strategies. For example, in some embodiments, intracellular delivery with the methods and systems of the disclosure may be over 100 times faster than the fastest mechanoporation methods currently available and over 1,000 times faster than most other transfection methods.

Second, some embodiments described herein may provide a flexible platform technology for ex vivo, intracellular delivery for a wide array of cell types and cargoes. Specifically, the magnitude and duration of the stretching force that the cells are subjected to is a predictable function of the microfluidic geometry, flow rate, polymer composition, and concentrations. Thus, in some embodiments, the stretching force may be controlled and optimized for different cell types.

Third, some embodiments described herein may provide a system that is capable of efficient and continuous operation. In some embodiments, because the cells are stretched by fluid forces alone, the microfluidic constriction (e.g., the micro-channel hydraulic diameter or cross-section) can be much larger than the diameter of the cells. In some embodiments, the devices and systems of the disclosure are, therefore, robust to failure by clogging or fouling, which is a major pitfall for scaling up existing mechanoporation strategies that use surface contact.

Fourth, some embodiments described herein may provide a device that is easily fabricated and able to be manufactured using a variety of methods. In some embodiments, the device does not require small geometries or high pressures and is compatible with most commercial manufacturing techniques including injection molding, hot embossing, and 3D printing.

Fifth, some embodiments described herein may provide methods and systems capable of delivery large cargoes (e.g., cargoes exceeding 25 nm in size) at high efficiencies (e.g., about 80% or more) and diverse nanoscale cargoes (e.g., regardless of electrostatic charge) while retaining high cell viability (e.g., about 80% or more). Thus, in some embodiments, the methods and systems provided herein are not limited to the delivery of smaller cargoes (e.g., nucleic acids) and can delivery larger cargoes (e.g., protein complexes such as RNP). In some embodiments, the methods and systems provided herein can provide contact-free, intracellular delivery of unmodified proteins directly to the cytosol of a cell.

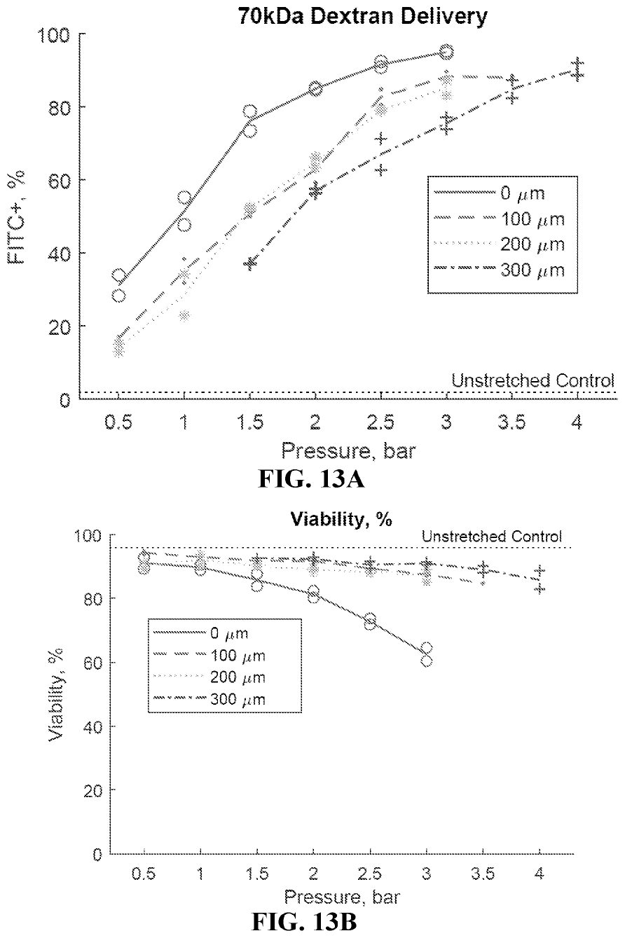

Sixth, some embodiments described herein may provide microfluidic methods and systems capable of processing very high cell densities (e.g., up to 1×10⁸ cells/mL, as shown in FIG. 12A). This is a significant advantage because current mechanoporation strategies require cells to be dilute (e.g. 5×10⁶ cells/ml or even lower). Therefore, in some embodiments, the microfluidic methods and systems of the disclosure are cost-effective as current mechanoporation methods require much larger amounts of expensive cargo per cell.

Seventh, some embodiments described herein can provide contact-less, intracellular delivery to a cell or particle that induces the formation of temporary, short-term pores that may seal within about 5 seconds or less. Specifically, the methods provided herein prevent and/or expose the cells or particles to less damage compared to other mechanoporation and/or electroporation methods. For example, the methods described may result in reduced or non-existent: organelle damage (e.g., mitochondrial damage), ion and pH imbalance, calcium influx, ATP depletion, and cell necrosis. Furthermore, the methods described do not aerosolize the cell suspension into airborne droplets as other methods (e.g., ultrasonic methods) do, which may lead to reduced cell recovery. Thus, in some embodiments, the methods provided herein may achieve intracellular delivery of payloads with minimal damage to cells or particles and increased cell recovery or a higher cell yields compared to other mechanoporation and/or electroporation methods.

Potential applications of viscoelastic cell stretching and mechanoporation methods and systems disclosed herein include, but are not limited to: 1) gene editing (e.g., gene knockouts, nucleic acid base editing, gene insertion) that may be used to develop cell therapies (e.g., T cell therapy, cell-based vaccines), 2) modulation of cellular pathways (e.g., DNA repair, differentiation of cells, activation of cells, reprogramming of cells) that may be used to in biomanufacturing applications (e.g., immunotherapy, vaccines, viral vectors), 3) deployment of intracellular sensors and/or actuators (e.g., cell labeling and/or manipulation) that may be used in diagnostics and cell analysis (e.g., single-cell studies, cellular diagnostics, disease modeling).

Methods Provided herein are methods of intracellular delivery to one or more cells, which include providing a substrate defining a micro-channel in fluid communication with a first chamber and optionally in fluid communication with a second chamber, and driving a cell suspension through the micro-channel, thereby: i) causing the one or more cells to be stretched along a direction of flow and ii) inducing a formation of one or more temporary pores in a membrane of the one or more cells. In some embodiments, the methods include providing a substrate defining a micro-channel that connects a first chamber and a second chamber. In some embodiments, the methods include providing a substrate defining a micro-channel extending between a first chamber and a second chamber.

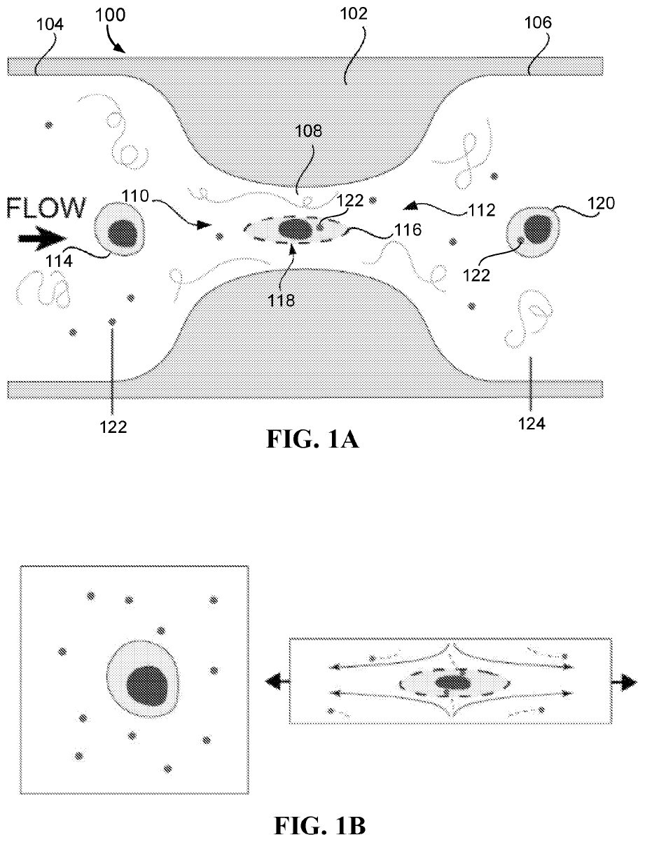

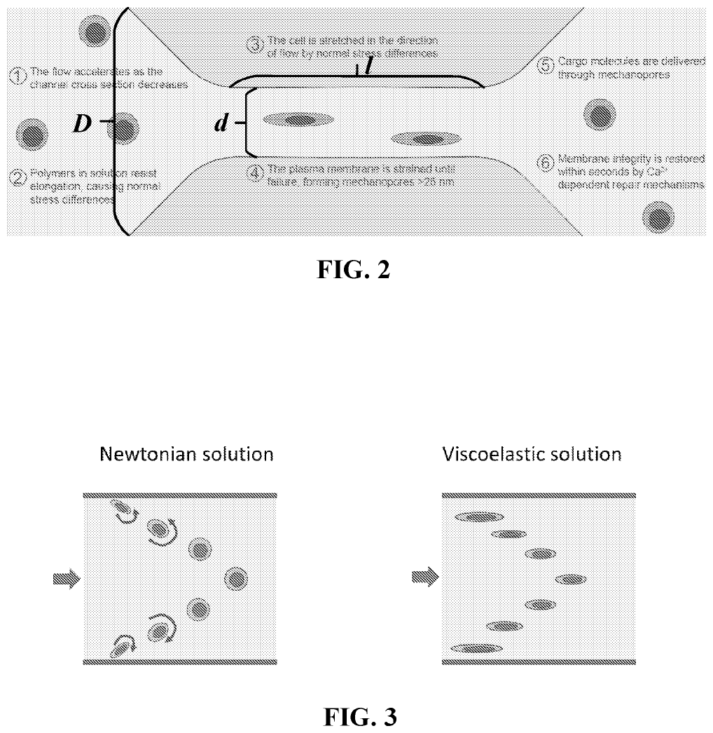

FIGS. 1A, 1B, and 2 are a schematics showing an exemplary method of intracellular delivery. Referring to FIG. 1A, the method includes providing a microfluidic chip 100 that includes a substrate 102, which defines a first chamber 104 and a second chamber 106. The substrate 102 further defines a micro-channel 108 that is positioned between and connects the first and second chambers 104, 106. The cell suspension containing an intact cell 114, the substance or cargo 122 to be delivered, and a polymer 124 is accelerated towards a first opening 110 of the micro-channel 108 from left to right in the direction of the arrow labeled “flow” The fluid flow accelerates as the cross-section (e.g., hydraulic diameter) of the chamber and micro-channel decreases. As shown in FIG. 2, the first and second chambers are tapered such that the hydraulic diameter D of the first and second chambers gradually narrows prior to intersecting an opening of the micro-channel having a hydraulic diameter d. In some embodiments, the first and second chambers are not tapered (e.g., the first and second chambers have a sharp intersection at 90-degree angles).

Referring back to FIG. 1A, the intact cell 114 is then pushed by the fluid flow into the micro-channel 108 through the first opening 110. While in the micro-channel 108, the intact cell 114 becomes a stretched cell 116 as it is stretched in the direction of flow by normal stress differences caused by a resistance of elongation of the polymer in the cell suspension, as shown in FIG. 1B. The cell membrane is then stretched until the onset of mechanoporation (i.e., until the formation temporary pores 118). The formation of temporary pores 118 enables the substance or cargo 122 to be delivered through these temporary pores 118 and into a cytoplasm (e.g., a cytosol) of the cell prior to exiting the micro-channel 108 through the second opening 112. As a result, a loaded cell 120 containing the substance or cargo 122 exits the micro-channel 108 and enters the second chamber 106 where it is then directed to an outlet of the microfluidic device to be collected. The loaded cell 120 restores its cell membrane integrity within a few seconds (e.g., less than about five seconds) and the temporary pores 118 are sealed. In some embodiments, the loaded cell 120 uses a calcium-dependent repair mechanism to restore its cell membrane integrity within a few seconds.

Referring back to FIG. 1B, when a fluid containing dilute polymers (e.g., a biopolymer such as hyaluronic acid) is pumped through a constriction, normal stress differences (akin to tension in solid mechanics) arise along streamlines. These viscoelastic forces are much larger than shear forces. Cells in the flow are therefore stretched in the direction of flow without rotation and without contact with a surface. Areal strain of the plasma membrane is highest along an equatorial band, which is also where the surrounding extensional flow impinges onto the cell surface. In some embodiments, the cells are stretched the most at a circumferential region around or near a center of each cell (e.g. the equatorial band or a “waist” of the cell). Furthermore, the driving of the cell suspension through the micro-channel causes advection (i.e., the movement of a mass of fluid) or molecular diffusion of the cell suspension toward this circumferential region. As a result, this advection or molecular diffusion causes the substance or cargo to be delivered through the temporary pores and into a cytosol of the cells. In some embodiments, the majority of the temporary pores (e.g., at least about 50%, 60%, 70%, 80%, 90%, 95%, or 99% of the temporary pores) are formed along the circumferential region around or near a center of each cell (e.g. the equatorial band or a “waist” of the cell).

By using the viscoelastic fluid forces that emerge during flow acceleration, the cells in the cell suspension are stretched without rotation anywhere in the flow and without contacting any surface. In some embodiments, the cells do not contact a surface of the micro-channel or of another cell, which may advantageously prevent fouling or damage to the cell. In some embodiments, the driving of the cell suspension through the micro-channel creates a viscoelastic extensional flow. In some embodiments, the viscoelastic extensional flow is a uniaxial extensional flow, a biaxial extensional flow, or a combination thereof.

In some embodiments, the cell suspension is driven through the micro-channel at a volumetric flow rate ranging from about 0.1 ml/minute to about 20 ml/minute (e.g., about 0.1 ml/min to about 0.5 ml/min, about 0.5 ml/min to about 1 ml/min, about 0.1 ml/min to about 1 ml/min, about 0.1 ml/min to about 3 ml/min, about 0.1 ml/min to about 4 ml/min, about 0.1 ml/min to about 5 ml/min, about 0.1 ml/min to about 6 ml/min, about 0.1 ml/min to about 7 ml/min, about 0.1 ml/min to about 8 ml/min, about 0.1 ml/min to about 9 ml/min, about 0.1 ml/min to about 10 ml/min, about 0.1 ml/min to about 15 ml/min, about 0.1 ml/min to about 20 ml/min, about 1 ml/minute to about 2 ml/minute, about 1 ml/minute to about 3 ml/minute, about 1 ml/minute to about 4 ml/minute, about 1 ml/minute to about 5 ml/minute, about 1 ml/minute to about 6 ml/minute, about 1 ml/minute to about 7 ml/minute, about 1 ml/minute to about 8 ml/minute, about 1 ml/minute to about 9 ml/minute, about 1 ml/minute to about 10 ml/minute, about 1 ml/minute to about 3 ml/minute). In some embodiments, the cell suspension is driven through the micro-channel at a volumetric flow rate ranging from about 1 ml/minute to about 3 ml/minute.

In some embodiments, the cell suspension is driven in the absence of a flow singularity. For example, the micro-channel and the chambers are microfabricated such that there are no features (e.g., indentations, protrusions, cavities, or the like) present on their surfaces that could create a stagnation point. In some embodiments, the micro-channel and the chambers are microfabricated such that there are no junctions (e.g., a T-junction, a Y-junction, a cross-shape, or the like) in the micro-channel geometry that could create a stagnation point.

The presence of a flow singularity during the viscoelastic mechanoporation is undesirable because it would require cells to be individually and sequentially trapped in the stagnation point. As a result, the cell suspension would be required to be dilute (e.g., about 1 million cells per mL or lower), thereby requiring a large amount of cargo molecule per cell. Since cells must pass individually through the stagnation point, throughput would also be inherently limited to 1-2 million cells per minute. Finally, the duration of the cell entrapment in the flow singularity is uncontrolled and stochastic. Also, shear gradients are maximally high near these stagnation points, leading to highly heterogeneous stretching forces with respect to cell size. As a result, the magnitude and duration of the stretching force is highly variable, leading to heterogeneous delivery efficiency and viability.

In some embodiments, the cell suspension is driven through the micro-channel at a volumetric flow rate resulting in a maximum Reynolds number of a flow ranging from about 10 to about 100 (e.g., about 10 to about 20, about 10 to about 30, about 10 to about 40, about 10 to about 50, about 10 to about 60, about 10 to about 70, about 10 to about 80, about 10 to about 90, about 10 to about 100, about 25 to about 50, about 25 to about 75, about 25 to about 100, about 50 to about 75, or about 50 to about 100).

The Weissenberg number is a dimensionless number used in the study of viscoelastic flows. This dimensionless number compares the elastic forces to the viscous forces in a fluid flow. The Weissenberg relates to the maximum normal stress differences in the flow (e.g., the stress on the cells in the cell delivery suspension). In some embodiments, the cell suspension is driven through the micro-channel at a volumetric flow rate resulting in a Weissenberg number ranging from about 1 to about 100 (e.g., about 1 to about 2, about 1 to about 3, about 1 to about 4, about 1 to about 5, about 1 to about 6, about 1 to about 7, about 1 to about 8, about 1 to about 9, about 1 to about 10, about 1 to about 15, about 1 to about 20, about 1 to about 30, about 1 to about 40, about 1 to about 50, about 1 to about 75, about 10 to about 20, about 10 to about 30, about 10 to about 40, about 10 to about 50, about 10 to about 60, about 10 to about 70, about 10 to about 80, about 10 to about 90, about 10 to about 100, about 25 to about 50, about 25 to about 75, about 25 to about 100, about 50 to about 60, about 50 to about 70, about 50 to about 75, about 50 to about 80, about 50 to about 90, or about 50 to about 100).

In some embodiments, the substrate 102 is a glass substrate. In some embodiments, the first and second chambers 104, 106 and the micro-channel 108 are composed of poly(dimethyl)siloxane (PDMS). The first and second chambers 104, 106 and the micro-channel 108 can be fabricated using PDMS microfabrication techniques known in the art. For example, liquid PDMS can be mixed with a cross-linking agent, poured into a micro-structured mold, and heated to obtain a solid, elastomeric replica of the mold. In some embodiments, the first and second chambers 104, 106 and the micro-channel 108 can be fabricated using alternative techniques or manufacturing methods including, but not limited to, stereolithography three-dimensional (3D) printing, micro-machining, injection molding, nanolithography, and electrical discharge machining (EDM).

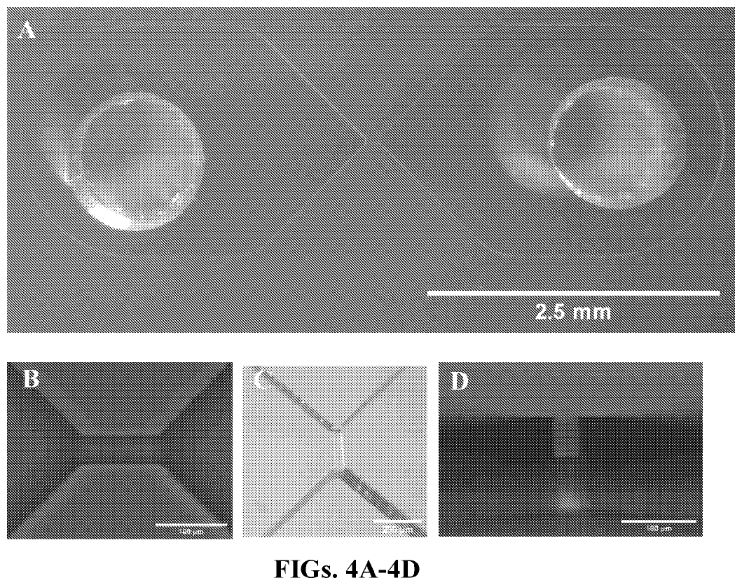

FIG. 3 is a schematic illustrating the behavior of cells suspended in a Newtonian solution or suspension and in a viscoelastic solution or suspension. Uneven mechanoporation for cells occurs when using a Newtonian solution or suspension with the methods and systems disclosed herein. Shear stress, which emerges from the gradient in the flow velocity due to wall drag, causes cell rotation and results in this undesired, uneven mechanoporation for cells at different distances from the wall. Contrastingly, in a viscoelastic solution or suspension under extensional flow, normal stress differences arise across all streamlines, and are generally much larger than shear stresses. As a result, all cells are stretched without rotation.

In some embodiments, the cell suspension has one or more viscoelastic properties. In some embodiments, the cell suspension is not a Newtonian fluid. In some embodiments, the cell suspension is accelerated while it is driven through the micro-channel. In some embodiments, the cell suspension is driven through the micro-channel by applying a driving pressure ranging from about 20 millibar (mbar) to about 7 bar (e.g., about 20 mbar to about 40 mbar, about 40 mbar to about 60 mbar, about 60 mbar to about 80 mbar, about 80 mbar to about 100 mbar, about 100 mbar to about 150 mbar, about 150 mbar to about 200 mbar, about 200 mbar to about 250 mbar, about 250 mbar to about 300 mbar, about 300 mbar to about 350 mbar, about 350 mbar to about 400 mbar, about 400 mbar to about 450 mbar, about 450 mbar to about 500 mbar, about 500 mbar to about 550 mbar, about 550 mbar to about 600 mbar, about 600 mbar to about 650 mbar, about 650 mbar to about 700 mbar, about 700 mbar to about 750 mbar, about 750 mbar to about 800 mbar, about 800 mbar to about 850 mbar, 850 mbar to about 900 mbar, about 900 mbar to about 950 mbar, about 950 mbar to about 1 bar, about 1 bar to about 2 bar, about 2 bar to about 3 bar, about 3 bar to about 4 bar, about 4 bar to about 5 bar, about 5 bar to about 6 bar, about 6 bar to about 7 bar, about 1 bar to about 3 bar, about 1 bar to about 4 bar, about 1 bar to about 5 bar, about 1 bar to about 6 bar, about 1 bar to about 7 bar, about 2 bar to about 3 bar, about 2 bar to about 4 bar, about 2 bar to about 5 bar, about 2 bar to about 6 bar, about 2 bar to about 7 bar, about 3 bar to about 4 bar, about 3 bar to about 5 bar, about 3 bar to about 6 bar, about 3 bar to about 7 bar, about 20 mbar to about 50 mbar, about 20 mbar to about 100 mbar, about 20 mbar to about 200 mbar, about 20 mbar to about 300 mbar, about 20 mbar to about 400 mbar, about 20 mbar to about 500 mbar, about 20 mbar to about 600 mbar, about 20 mbar to about 700 mbar, about 20 mbar to about 800 mbar, about 20 mbar to about 900 mbar, about 20 mbar to about 1 bar, about 20 mbar to about 2 bar, about 20 mbar to about 3 bar, about 20 mbar to about 4 bar, about 20 mbar to about 5 bar, or about 20 mbar to about 6 bar). In some embodiments, the cell suspension is driven through the micro-channel by applying a driving pressure ranging from about 1 bar to about 3 bar.

In some embodiments, the one or more temporary pores have a diameter ranging from about 1 nm to about 100 nm (e.g., about 1 nm to about 3 nm, about 1 nm to 5 nm, about 1 nm to about 10 nm, about 1 nm to about 15 nm, about 1 nm to about 20 nm, about 1 nm to about 25 nm, about 1 nm to about 30 nm, about 1 nm to about 40 nm, about 1 nm to about 50 nm, about 1 nm to about 60 nm, about 1 nm to about 70 nm, about 1 nm to about 80 nm, about 1 nm to about 90 nm, about 1 nm to about 95 nm, about 5 nm to about 10 nm, about 5 nm to about 15 nm, about 5 nm to about 20 nm, about 5 nm to about 25 nm, about 5 nm to about 30 nm, about 5 nm to about 40 nm, about 5 nm to about 50 nm, about 5 nm to about 60 nm, about 5 nm to about 70 nm, about 5 nm to about 80 nm, about 5 nm to about 90 nm, about 5 nm to about 100 nm, about 10 nm to about 15 nm, about 10 nm to about 20 nm, about 10 nm to about 25 nm, about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 100 nm, about 15 nm to about 20 nm, about 15 nm to about 25 nm, about 15 nm to about 30 nm, about 15 nm to about 40 nm, about 15 nm to about 50 nm, about 15 nm to about 60 nm, about 15 nm to about 70 nm, about 15 nm to about 80 nm, about 15 nm to about 90 nm, about 15 nm to about 100 nm, about 20 nm to about 25 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 25 nm to about 30 nm, about 25 nm to about 40 nm, about 25 nm to about 50 nm, about 25 nm to about 60 nm, about 25 nm to about 70 nm, about 25 nm to about 80 nm, about 25 nm to about 90 nm, about 25 nm to about 100 nm, about 50 nm to about 60 nm, about 50 nm to about 70 nm, about 50 nm to about 80 nm, about 50 nm to about 90 nm, or about 50 nm to about 100 nm). In some embodiments, the one or more temporary pores have a diameter of about 25 nm.

In some embodiments, the one or more temporary pores are sealed by the one or more cells within about 1 second to about 20 seconds (e.g., about 1 second to about 2 seconds, about 1 second to about 2 seconds, about 1 second to about 3 seconds, about 1 second to about 4 seconds, about 1 second to about 5 seconds, about 1 second to about 6 seconds, about 1 second to about 7 seconds, about 1 second to about 8 seconds, about 1 second to about 9 seconds, about 1 second to about 10 seconds, about 1 second to about 11 seconds, about 1 second to about 12 seconds, about 1 second to about 13 seconds, about 1 second to about 14 seconds, about 1 second to about 15 seconds, about 1 second to about 15 seconds, about 1 second to about 16 seconds, about 1 second to about 17 seconds, about 1 second to about 18 seconds, about 1 second to about 19 seconds, about 1 second to about 20 seconds, about 5 seconds to about 10 seconds, about 5 seconds to about 15 seconds, about 5 seconds to about 20 seconds, about 10 seconds to about 15 seconds, or about 10 seconds to about 20 seconds) after their formation. In some embodiments, the one or more temporary pores are sealed by the one or more cells within about 1 second to about 10 seconds after their formation. In some embodiments, the presence of calcium ions in the cell suspension induces the temporary pores to seal within about 1 second to about 20 seconds. In some embodiments, the absence of calcium ions in the cell suspension induces the temporary pores to seal within about 2 seconds to about 10 seconds. In some embodiments, the absence of calcium ions in the cell suspension induces the temporary pores to seal within about 5 seconds. In some embodiments, the absence of calcium ions in the cell suspension induces the temporary pores to seal within about 5 seconds or less.

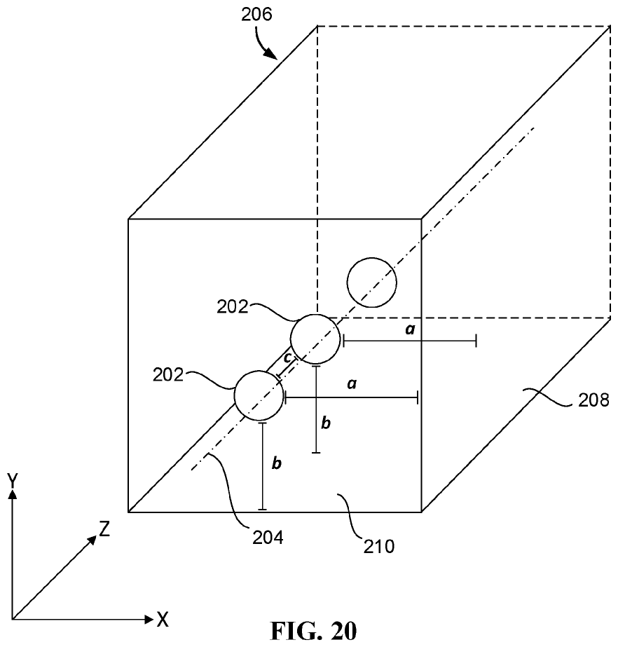

In some embodiments, the methods further include focusing the one or more cells into a localized fluid flow path optionally at or near a center of the micro-channel or near a wall of the micro-channel within a moving fluid. In some embodiments, the methods further include focusing the one or more cells into a localized fluid flow path within the moving fluid at any location within the micro-channel where the cells are streamlined at a same distance within each other and at a same x, y coordinate within two-dimensional planes of the micro-channel. FIG. 20 is a schematic illustrating an exemplary localized fluid flow path. As shown in FIG. 20, the particles 202 are streamlined in a localized fluid flow path 204 within the micro-channel 206. The particles 202 are aligned in such a way that each particle 202: i) is located at a same distance a along the x-axis, away from a side wall 208 of the micro-channel 206, ii) is located at a same distance b along the y-axis, away from a bottom surface 210 of the micro-channel 206, and iii) is located within a same distance c along the z-axis, away from each other. The exemplary localized fluid flow path 204 is illustrated as a line, but the localized fluid flow path can have any suitable dimensions (e.g., any suitable length, width, height, and/or hydraulic diameter). In some embodiments, particle 202 is a cell. In some embodiments, particle 202 is any type of particle described elsewhere herein.

In some embodiments, the particles 202 are aligned in such a way that each particle 202 is located at a distance a of about 0.5 μm to about 10 μm (e.g., about 0.5 μm to about 1 μm, about 0.5 μm to about 1.5 μm, about 0.5 μm to about 2 μm, about 0.5 μm to about 3 μm, about 0.5 μm to about 4 μm, about 0.5 μm to about 5 μm, about 0.5 μm to about 6 μm, about 0.5 μm to about 7 μm, about 0.5 μm to about 8 μm, about 0.5 μm to about 9 μm, about 1 μm to about 5 μm, about 1 μm to about 10 μm, or about 5 μm to about 10 μm) along the x-axis, away from a side wall 208 of the micro-channel 206.

In some embodiments, the particles 202 are aligned in such a way that each particle 202 is located at a distance b of about 5 μm to about 25 μm (e.g., about 5 μm to about 6 μm, about 5 μm to about 7 μm, about 5 μm to about 8 μm, about 5 μm to about 9 μm, about 5 μm to about 10 μm, about 5 μm to about 15 μm, about 5 μm to about 20 μm, about 5 μm to about 25 μm, about 10 μm to about 15 μm, about 10 μm to about 20 μm, about 10 μm to about 25 μm, about 15 μm to about 20 μm, or about 15 μm to about 25 μm) along the y-axis, away from a bottom surface 210 or a top surface of the micro-channel 206.

In some embodiments, the particles 202 are aligned in such a way that each particle 202 is located at a distance c of about 5 μm to about 100 μm (e.g., about 5 μm to about 6 μm, about 5 μm to about 7 μm, about 5 μm to about 8 μm, about 5 μm to about 9 μm, about 5 μm to about 10 μm, about 5 μm to about 15 μm, about 5 μm to about 20 μm, about 5 μm to about 25 μm, about 5 μm to about 30 μm, about 5 μm to about 35 μm, about 5 μm to about 40 μm, about 5 μm to about 45 μm, about 5 μm to about 50 μm, about 5 μm to about 55 μm, about 5 μm to about 60 μm, about 5 μm to about 65 μm, about 5 μm to about 70 μm, about 5 μm to about 75 μm, about 5 μm to about 80 μm, about 5 μm to about 85 μm, about 5 μm to about 90 μm, about 5 μm to about 95 μm, about 50 μm to about 75 μm, about 75 μm to about 100 μm, about 25 μm to about 50 μm, about 25 μm to about 75 μm, or about 25 μm to about 100 μm) along the z-axis, away from each other. In some embodiments, the distance c along the z-axis is dependent upon cell density.

In some embodiments, the volumetric flow rate at which the cell suspension is driven can be controlled such that it results in the formation of a localized flow path in the fluid that streamlines the cells to be within a same distance to each other and within a same distance to a surface of the micro-channel. The localized flow path defines a hydraulic diameter that is substantially equal to or slightly greater than a diameter of the cell. As a result, the cells in the cell suspension are focused into the localized flow path.

Furthermore, the addition of specified concentrations (e.g., micromolar concentrations or any of the other concentrations described elsewhere herein) of one or more polymers (e.g., hyaluronic acid (HA)) results in a fluid viscoelasticity that can be used to control the focal position of the particles at different Reynolds numbers (Re). Non-limiting aspects of inertio-elastic particle focusing methodologies are described in U.S. Pat. Nos. 10,307,760, 9,808,803, 9,895,694, and 9,610,582.

### Cell Suspensions

In some embodiments, the cell suspension includes one or more cells, a polymer, and the substance or agent to be delivered intracellularly.

### Cells

In some embodiments, the cells dispersed in the cell suspension are immune cells. Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells. In some embodiments, the cells dispersed in the cell suspension are T cells or Jurkat cells.

In some embodiments, the cells dispersed in the cell suspension can be derived from a cell culture grown in vitro. Samples derived from a cell culture can include one or more suspension cells which are anchorage-independent within the cell culture. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M.

Samples derived from a cell culture can include one or more adherent cells which grow on the surface of the vessel that contains the culture medium. Non-limiting examples of adherent cells include DU145 (prostate cancer) cells, H295R (adrenocortical cancer) cells, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCI60), vero (African green monkey Chlorocebus kidney epithelial cell line) cells, MC3T3 (embryonic calvarium) cells, GH3 (pituitary tumor) cells, PC12 (pheochromocytoma) cells, dog MDCK kidney epithelial cells, Xenopus A6 kidney epithelial cells, zebrafish AB9 cells, and Sf9 insect epithelial cells.

In some embodiments, the adherent cells are cells that correspond to one or more of the following cell lines: BT549, HS 578T, MCF7, MDA-MB-231, MDA-MB-468, T-47D, SF268, SF295, SF539, SNB-19, SNB-75, U251, Colo205, HCC 2998, HCT-116, HCT-15, HT29, KM12, SW620, 786-0, A498, ACHN, CAM, RXF 393, SN12C, TK-10, UO-31, A549, EKVX, HOP-62, HOP-92, NCI-H226, NCI-H23, NCI-H460, NCI-H522, LOX IMVI, M14, MALME-3M, MDA-MB-435, SK-, EL-2, SK-MEL-28, SK-MEL-5, UACC-257, UACC-62, IGROV1, OVCAR-3, OVCAR-4, OVCAR-5, OVCAR-8, SK-OV-3, NCI-ADR-RES, DU145, PC-3, DU145, H295R, HeLa, KBM-7, LNCaP, MCF-7, MDA-MB-468, PC3, SaOS-2, SH-SY5Y, T-47D, THP-1, U87, vero, MC3T3, GH3, PC12, dog MDCK kidney epithelial, Xenopus A6 kidney epithelial, zebrafish AB9, and Sf9 insect epithelial cell lines.

In some embodiments, the cell suspension includes cells at a concentration ranging from about 1 million cells/ml to about 200 million cells/ml (e.g., about 1 million cells/ml to about 3 million cells/ml, about 3 million cells/ml to about 10 million cells/ml, about 10 million cells/ml to about 15 million cells/ml, about 15 million cells/ml to about 20 million cells/ml, about 20 million cells/ml to about 25 million cells/ml, about 25 million cells/ml to about 30 million cells/ml, about 30 million cells/ml to about 35 million cells/ml, about 35 million cells/ml to about 40 million cells/ml, about 40 million cells/ml to about 45 million cells/ml, about 45 million cells/ml to about 50 million cells/ml, about 50 million cells/ml to about 55 million cells/ml, about 55 million cells/ml to about 60 million cells/ml, about 60 million cells/ml to about 65 million cells/ml, about 65 million cells/ml to about 70 million cells/ml, about 70 million cells/ml to about 75 million cells/ml, about 75 million cells/ml to about 80 million cells/ml, about 80 million cells/ml to about 85 million cells/ml, about 85 million cells/ml to about 90 million cells/ml, about 90 million cells/ml to about 95 million cells/ml, about 95 million cells/ml to about 100 million cells/ml, about 1 million cells/ml to about 50 million cells/ml, about 1 million cells/ml to about 100 million cells/ml, about 1 million cells/ml to about 150 million cells/ml, about 1 million cells/ml to about 200 million cells/ml, about 50 million cells/ml to about 75 million cells/ml, about 50 million cells/ml to about 100 million cells/ml, about 50 million cells/ml to about 125 million cells/ml, about 50 million cells/ml to about 150 million cells/ml, about 50 million cells/ml to about 175 million cells/ml, about 50 million cells/ml to about 200 million cells/ml, or more). In some embodiments, the cell suspension includes cells at a concentration of about 50 million cells/ml. In some embodiments, the cell suspension includes cells at a concentration ranging from about 1 million cells/ml to about 100 million cells/ml.

In some implementations, the viability of cells can be assessed via known methods known in the art, such as propidium iodide (PI) exclusion. PI is a membrane impermeant dye that is generally excluded from viable cells. It binds to double stranded DNA by intercalating between base pairs. PI is excited at 488 nm and emits at a maximum wavelength of 617 nm. In an example, cells that have been exposed to viscoelastic stretching and mechanoporation and have received a cargo can be contacted with PI to quantify the number of cells that exclude the dye (i.e., the viable cells). In some embodiments, the cell viability ranges from about 50% to about 99% (e.g., at least about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 99%). In some embodiments, the cell viability decreases with increasing pressure (e.g., the driving pressure used to drive the cell suspension through the micro-channel). In some embodiments, the cell viability decreases with increasing delivery efficiency. In some embodiments, the cell viability is dependent on the cell type that is subjected to the methods disclosed herein.

The delivery efficiency refers to the percentage of the substance or cargo that is successfully delivered to the cytosol of a cell after subjecting the cell to the methods disclosed herein. In an example, the delivery efficiency can be calculated by measuring and comparing a fluorescence intensity of a fluorescently-labeled cargo and a fluorescence intensity of a control condition (e.g., unstretched cells that are incubated with the same polymer and cargo substance for a similar amount of time and similar temperature), In some embodiments, the delivery efficiency ranges from about 50% to about 99% (e.g., at least about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 99%). In some embodiments, the delivery efficiency increases with increasing pressure. In some embodiments, the cell concentration of the cell suspension does not have an effect on the delivery efficiency. In some embodiments, the delivery efficiency is dependent on the cell type that is subjected to the methods disclosed herein. In some embodiments, the delivery efficiency of a cargo dispersed or dissolved in a cell suspension that includes a polymer (e.g., hyaluronic acid) is greater than the delivery efficiency of a cargo that is dispersed or dissolved in a cell suspension that does not include a polymer. In some embodiments, the delivery efficiency of a cargo dispersed or dissolved in a viscoelastic cell suspension (e.g., a suspension that exhibits one or more viscoelastic fluid properties) is greater than the delivery efficiency of a cargo that is dispersed or dissolved in a Newtonian cell suspension (e.g., a suspension that exhibits Newtonian fluid properties). In some embodiments, the delivery efficiency of a cargo dispersed or dissolved in a non-Newtonian cell suspension (e.g., a suspension that exhibits non-Newtonian fluid properties) is greater than the delivery efficiency of a cargo that is dispersed or dissolved in a Newtonian cell suspension.

### Polymers

In some embodiments, the cell suspension includes a polymer (e.g., a long-chain polymer or a long-chain branched polymer). In some embodiments, the polymer is a biopolymer. In some embodiments, the biopolymer is a polymer that includes one or more components that are naturally-derived or naturally occurring. In some embodiments, the biopolymer is a polymer that includes one or more components that are naturally-derived or naturally occurring and one or more components that are synthetically modified. In some embodiments, the biopolymer is a polysaccharide, a proteoglycan, or a polypeptide. In some embodiments, the polymer is a polymer produced by a cell of a living organism. In some embodiments, the polymer is a synthetic polymer. In some embodiments, the cell suspension includes hyaluronic acid (HA). In some embodiments, the cell suspension includes a polymer comprising poly(ethylene) glycol, poly(vinyl) pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate, polycaprolactone, cellulose, lignin, alginate, chitosan, starch, or any combination thereof. In some embodiments, the polymer (e.g., HA) is present in the cell suspension at a concentration ranging from about 0.001 mg/ml to about 4 mg/ml (e.g., about 0.001 mg/ml to about 0.005 mg/ml, about 0.005 mg/ml to about 0.010 mg/ml, about 0.001 mg/ml to about 0.01 mg/ml, about 0.01 mg/ml to about 0.050 mg/ml, about 0.050 mg/ml to about 0.1 mg/ml, about 0.01 mg/ml to about 0.1 mg/ml, about 0.001 mg/ml to about 0.05 mg/ml, about 0.001 mg/ml to about 0.1 mg/ml, about 0.1 mg/ml to about 0.5 mg/ml, about 0.5 mg/ml to about 1 mg/ml, about 0.5 mg/ml to about 2 mg/ml, about 0.5 mg/ml to about 3 mg/ml, about 0.5 mg/ml to about 4 mg/ml, about 0.001 mg/ml to about 0.5 mg/ml, about 0.001 mg/ml to about 1 mg/ml, about 0.01 mg/ml to about 0.5 mg/ml, about 0.01 mg/ml to about 1 mg/ml, about 1 mg/ml to about 1.5 mg/ml, about 1.5 mg/ml to about 2 mg/ml, about 2 mg/ml to about 2.5 mg/ml, about 2.5 mg/ml to about 3 mg/ml, about 3 mg/ml to about 3.5 mg/ml, about 3.5 mg/ml to about 4 mg/ml, about 0.001 mg/ml to about 2 mg/ml, about 0.01 mg/ml to about 2 mg/ml, about 0.1 mg/ml to about 2 mg/ml, about 1 mg/ml to about 2 mg/ml, about 0.001 mg/ml to about 3 mg/ml, about 0.01 mg/ml to about 3 mg/ml, about 0.1 mg/ml to about 3 mg/ml, about 1 mg/ml to about 3 mg/ml, about 0.001 mg/ml to about 3.5 mg/ml, about 0.01 mg/ml to about 4 mg/ml, about 0.1 mg/ml to about 4 mg/ml, about 1 mg/ml to about 4 mg/ml, about 2 mg/ml to about 4 mg/ml, or about 3 mg/ml to about 4 mg/ml). In some embodiments, HA is present in the cell suspension at a concentration of about 0.1 mg/ml to about 3 mg/ml. In some embodiments, HA is present in the cell suspension at a concentration of about 2 mg/ml to about 4 mg/ml. In some embodiments, HA is present in the cell suspension at a concentration of about 0.5 mg/ml to about 3 mg/ml. In some embodiments, HA is present in the cell suspension at a concentration of about 1 mg/ml.

In some embodiments, the cell suspension includes a polymer having a molecular mass ranging from about 0.1 megaDaltons (MDa) to about 2 MDa (e.g., about 0.1 MDa to about 0.5 MDa, about 0.1 MDa to about 0.2 MDa, about 0.1 MDa to about 0.3 MDa, about 0.1 MDa to about 0.4 MDa, about 0.1 MDa to about 0.5 MDa, about 0.1 MDa to about 0.6 MDa, about 0.1 MDa to about 0.7 MDa, about 0.1 MDa to about 0.8 MDa, about 0.1 MDa to about 0.9 MDa, about 0.5 MDa to about 1 MDa, about 0.1 MDa to about 1 MDa, about 1 MDa to about 1.5 MDa, about 1.5 MDa to about 2 MDa, about 1 MDa to about 2 MDa, about 0.5 MDa to about 2 MDa, or more). In some embodiments, the cell suspension includes a polymer having a molecular mass of about 1.5 MDa.

### Intracellular Cargoes

The substance or cargo dispersed, suspended, or diluted in the cell delivery suspension can include, but is not limited to, a protein, a nucleic acid, a nanomaterial, a drug, or any combination thereof. In some embodiments, substance or cargo dissolved, dispersed, suspended, or diluted in the cell delivery suspension can include, but is not limited to, naturally occurring, modified, or synthetic protein. In some embodiments, substance or cargo dissolved, dispersed, suspended, or diluted in the cell delivery suspension can include, but is not limited to, naturally occurring, modified, or synthetic nucleic acid. In some embodiments, the substance can include a naturally occurring, modified, or synthetic protein such as an enzyme, an endonuclease (e.g., a Clustered Regularly Interspaced Short Palindromic Repeats (CRISPR)-associated (Cas) endonuclease), a transcription factor (e.g., reprogramming factor), an antibody (e.g., a single variable domain on a heavy chain (VHH) antibody, a Nanobody®), a peptide, a transcription activator-like effector nuclease (TALEN), a zinc finger nuclease (ZFNs), a DNA repair factor, a ribonucleoprotein, a lipid-protein complex, or any combination thereof. In some examples, ex vivo, intracellular delivery of DNA-editing enzymes (e.g., Cas9, TALEN, and/or ZFNs) to cells (e.g., immune cells) using the methods disclosed herein can result in successful gene insertion, nucleic acid base editing, and gene knockouts, thereby enabling the production of cell therapies (e.g., T cell therapies and/or cell-based vaccines). In some embodiments, the substance reprogramming factors such as Oct4, Sox2, Klf4, cMyc, or any combination thereof. In some embodiments, the intracellular delivery of transcription factors to cells (e.g., somatic cells) using the methods disclosed herein can enable the generation of induced pluripotent stem cells (iPSCs) at a high-throughput scale.

In some embodiments, the substance can include a naturally occurring, modified, or synthetic nucleic acid such as, but not limited to, deoxyribonucleic acid (DNA), ribonucleic acid (RNA), viral vectors, chromosomes, aptamers, nucleosomes, or any combination thereof. Non-limiting examples of nucleic acids include DNA such as genomic DNA, methylated DNA, specific methylated DNA sequences, fragmented DNA, mitochondrial DNA, and RNA/DNA hybrids.

Non-limiting examples of nucleic acids also include RNA such as various types of coding and non-coding RNA. Examples of the different types of RNA include messenger RNA (mRNA), ribosomal RNA (rRNA), transfer RNA (tRNA), microRNA (miRNA), viral RNA, CRISPR RNA (crRNA), trans-activating CRISPR RNA (tracrRNA), single guide RNA (sgRNA), and crRNA/tracrRNA hybrid. The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments, the DNA sequence is a CRISPR nucleic acid sequence. The substance can include a nucleic acid capable of functioning as a component of a gene editing reaction, such as, for example, CRISPR-based gene editing. In some embodiments, the substance is a RNA-Cas9 ribonucleoprotein (RNP) complex.

In some embodiments, the substance or cargo to be delivered intracellularly using the methods and systems disclosed herein can include a nanomaterial or a nanoparticle. Non-limiting examples of nanomaterials or nanoparticles that can be delivered intracellularly using the methods and systems of the disclosure include quantum dots, plasmonic nanoparticles, metallic nanoparticles, polymeric nanoparticles, liposomes, lipid nanoparticles, exosomes, or any combination thereof. In some embodiments, the nanomaterial or nanoparticle has a size (e.g. a diameter) of less than about 300 nm. In some embodiments, the nanomaterial or nanoparticle can have a diameter of between about 2 nm to about 200 nm (e.g., between about 10 nm to about 30 nm, between about 5 nm to about 25 nm, between about 10 nm to about 25 nm, between about 15 nm to about 25 nm, between about 20 nm and about 25 nm, between about 25 nm to about 50 nm, between about 50 nm and about 200 nm, between about 70 nm and about 200 nm, between about 80 nm and about 200 nm, between about 100 nm and about 200 nm, between about 140 nm to about 200 nm, and between about 150 nm to about 200 nm).

In some embodiments, the nanomaterial or nanoparticle can be spherical or ellipsoidal, or can have an amorphous shape. In some embodiments, the nanomaterial or nanoparticle can be magnetic (e.g., include a core of a magnetic material). In some embodiments, the magnetic material or particle can contain a diamagnetic, paramagnetic, superparamagnetic, or ferromagnetic material that is responsive to a magnetic field.

In some embodiments, the nanomaterial or nanoparticle can contain, in part, a core and/or a shell of containing a polymer (e.g., poly(lactic-co-glycolic acid)). Skilled practitioners will appreciate that any number of art known materials can be used to prepare nanoparticles, including, but are not limited to, gums (e.g., Acacia, Guar), chitosan, gelatin, sodium alginate, and albumin. Additional polymers that can be used to generate the nanomaterial or nanoparticle to be dispersed in the cell solution are known in the art. For example, polymers that can be used to generate the nanomaterial or nanoparticle include, but are not limited to, cellulosics, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate and polycaprolactone.

In some embodiments, the substance or cargo to be delivered intracellularly using the methods and systems disclosed herein can include catalysts, polysaccharides, viral particles, bacteria, organelles (e.g., mitochondria, endosomes, nuclei, ribosomes, endoplasmic reticula, or the like), contrast agents (e.g., magnetic resonance imaging (MRI) contrast agents such as gadolinium agents, iron oxide nanoparticles, superparamagnetic iron-platinum nanoparticles, manganese chelates, perfluorocarbons, barium sulfate agents, or the like). In some embodiments, the substance or cargo to be delivered intracellularly using the methods and systems disclosed herein can include molecular probes including a molecular recognition agent that can be conjugated to a detection element such as a fluorophore, a dye, or the like. Non-limiting examples of drugs that can be dissolved, dispersed, suspended, or diluted in the cell delivery suspension can include an anti-inflammatory, an antibiotic, an analgesic, an anti-viral drug, an anti-cancer drug, an immunosuppressant, an anticoagulant, and an anti-fungal drug.

In some embodiments, the substance or cargo has a molecular mass ranging from about 0.1 megaDaltons (MDa) to about 2 MDa (e.g., about 0.1 MDa to about 0.5 MDa, about 0.1 MDa to about 0.2 MDa, about 0.1 MDa to about 0.3 MDa, about 0.1 MDa to about 0.4 MDa, about 0.1 MDa to about 0.5 MDa, about 0.1 MDa to about 0.6 MDa, about 0.1 MDa to about 0.7 MDa, about 0.1 MDa to about 0.8 MDa, about 0.1 MDa to about 0.9 MDa, about 0.5 MDa to about 1 MDa, about 0.1 MDa to about 1 MDa, about 1 MDa to about 1.5 MDa, about 1.5 MDa to about 2 MDa, about 1 MDa to about 2 MDa, about 0.5 MDa to about 2 MDa, or more).

In some embodiments, the substance or cargo has a size (e.g., a diameter) ranging from about 1 nm to about 100 nm or more (e.g., about 1 nm to about 5 nm, about 5 nm to about 10 nm, about 10 nm to about 15 nm, about 15 nm to about 20 nm, about 20 nm to about 25 nm, about 1 nm to about 5 nm, about 1 nm to about 10 nm, about 1 nm to about 15 nm, about 1 nm to about 20 nm, about 5 nm to about 15 nm, about 5 nm to about 20 nm, about 5 nm to about 25 nm, about 10 nm to about 20 nm, about 10 nm to about 25 nm, about 15 nm to about 25 nm, about 1 nm to about 25 nm, about 1 nm to about 30 nm, about 1 nm to about 40 nm, about 1 nm to about 50 nm, about 1 nm to about 60 nm, about 1 nm to about 70 nm, about 1 nm to about 80 nm, about 1 nm to about 90 nm, about 1 nm to about 95 nm, about 20 nm to about 25 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 25 nm to about 30 nm, about 25 nm to about 40 nm, about 25 nm to about 50 nm, about 25 nm to about 60 nm, about 25 nm to about 70 nm, about 25 nm to about 80 nm, about 25 nm to about 90 nm, about 25 nm to about 100 nm, about 50 nm to about 60 nm, about 50 nm to about 70 nm, about 50 nm to about 80 nm, about 50 nm to about 90 nm, about 50 nm to about 100 nm, or more). In some embodiments, the substance or cargo has a size (e.g., a diameter) of about 25 nm. In some embodiments, the substance or cargo has a size (e.g., a diameter) of about 13 nm.

In some embodiments, the substance or cargo is present in the cell suspension at a concentration ranging from about 0.0001 mg/ml to about 4 mg/ml (e.g., about 0.0001 mg/ml to about 0.0005 mg/ml, about 0.0001 mg/ml to about 0.001 mg/ml, about 0.0001 mg/ml to about 0.01 mg/ml, about 0.0001 mg/ml to about 0.1 mg/ml, about 0.0001 mg/ml to about 1 mg/ml, about 0.001 mg/ml to about 0.005 mg/ml, about 0.005 mg/ml to about 0.010 mg/ml, about 0.001 mg/ml to about 0.01 mg/ml, about 0.01 mg/ml to about 0.050 mg/ml, about 0.050 mg/ml to about 0.1 mg/ml, about 0.01 mg/ml to about 0.1 mg/ml, about 0.001 mg/ml to about 0.05 mg/ml, about 0.001 mg/ml to about 0.1 mg/ml, about 0.1 mg/ml to about 0.5 mg/ml, about 0.5 mg/ml to about 1 mg/ml, about 0.5 mg/ml to about 2 mg/ml, about 0.5 mg/ml to about 3 mg/ml, about 0.5 mg/ml to about 4 mg/ml, about 0.001 mg/ml to about 0.5 mg/ml, about 0.001 mg/ml to about 1 mg/ml, about 0.01 mg/ml to about 0.5 mg/ml, about 0.01 mg/ml to about 1 mg/ml, about 1 mg/ml to about 1.5 mg/ml, about 1.5 mg/ml to about 2 mg/ml, about 2 mg/ml to about 2.5 mg/ml, about 2.5 mg/ml to about 3 mg/ml, about 3 mg/ml to about 3.5 mg/ml, about 3.5 mg/ml to about 4 mg/ml, about 0.001 mg/ml to about 2 mg/ml, about 0.01 mg/ml to about 2 mg/ml, about 0.1 mg/ml to about 2 mg/ml, about 1 mg/ml to about 2 mg/ml, about 0.001 mg/ml to about 3 mg/ml, about 0.01 mg/ml to about 3 mg/ml, about 0.1 mg/ml to about 3 mg/ml, about 1 mg/ml to about 3 mg/ml, about 0.001 mg/ml to about 3.5 mg/ml, about 0.01 mg/ml to about 4 mg/ml, about 0.1 mg/ml to about 4 mg/ml, about 1 mg/ml to about 4 mg/ml, about 2 mg/ml to about 4 mg/ml, or about 3 mg/ml to about 4 mg/ml). In some embodiments, the substance or cargo is present in the cell suspension at a concentration of about 0.2 mg/ml.

### Additional Cell Suspension Components

In some embodiments, the cell suspension includes one or more ions including, but not limited to, calcium ions, sodium ions, magnesium ions, chloride ions, potassium ions, or any combination thereof. In some embodiments, the cell suspension includes calcium ions or calcium chloride. In some embodiments, the cell suspension includes one or more ions (e.g., calcium ions or calcium chloride) at a concentration of about 1 μM to about 5 mM (e.g., about 1 μM to about 100 μM, about 1 μM to about 200 μM, about 1 μM to about 300 μM, about 1 μM to about 400 μM, about 1 μM to about 500 μM, about 1 μM to about 600 μM, about 1 μM to about 700 μM, about 1 μM to about 800 μM, about 1 μM to about 900 μM, about 1 μM to about 1 mM, about 1 μM to about 2 mM, about 1 μM to about 3 mM, about 1 μM to about 4 mM, about 100 μM to about 200 μM, about 100 μM to about 300 μM, about 100 μM to about 400 μM, about 100 μM to about 500 μM, about 100 μM to about 600 μM, about 100 μM to about 700 μM, about 100 μM to about 800 μM, about 100 μM to about 900 μM, about 100 μM to about 1 mM, about 100 μM to about 2 mM, about 100 μM to about 3 mM, about 100 μM to about 4 mM, about 100 μM to about 5 mM, about 200 μM to about 300 μM, about 200 μM to about 400 μM, about 200 μM to about 500 μM, about 200 μM to about 600 μM, about 200 μM to about 700 μM, about 200 μM to about 800 μM, about 200 μM to about 900 μM, about 200 μM to about 1 mM, about 200 μM to about 2 mM, about 200 μM to about 3 mM, about 200 μM to about 4 mM, about 200 μM to about 5 mM, about 300 μM to about 400 μM, about 300 μM to about 500 μM, about 300 μM to about 600 μM, about 300 μM to about 700 μM, about 300 μM to about 800 μM, about 300 μM to about 900 μM, about 300 μM to about 1 mM, about 300 μM to about 2 mM, about 300 μM to about 3 mM, about 300 μM to about 4 mM, about 300 μM to about 5 mM, about 400 μM to about 500 μM, about 400 μM to about 600 μM, about 400 μM to about 700 μM, about 400 μM to about 800 μM, about 400 μM to about 900 μM, about 400 μM to about 1 mM, about 400 μM to about 2 mM, about 400 μM to about 3 mM, about 400 μM to about 4 mM, about 400 μM to about 5 mM, about 500 μM to about 600 μM, about 500 μM to about 700 μM, about 500 μM to about 800 μM, about 500 μM to about 900 μM, about 500 μM to about 1 mM, about 500 μM to about 2 mM, about 500 μM to about 3 mM, about 500 μM to about 4 mM, about 500 μM to about 5 mM, about 600 μM to about 700 μM, about 600 μM to about 800 μM, about 600 μM to about 900 μM, about 600 μM to about 1 mM, about 600 μM to about 2 mM, about 600 μM to about 3 mM, about 600 μM to about 4 mM, about 600 μM to about 5 mM, about 700 μM to about 800 μM, about 700 μM to about 900 μM, about 700 μM to about 1 mM, about 700 μM to about 2 mM, about 700 μM to about 3 mM, about 700 μM to about 4 mM, about 700 μM to about 5 mM, about 800 μM to about 900 μM, about 800 μM to about 1 mM, about 800 μM to about 2 mM, about 800 μM to about 3 mM, about 800 μM to about 4 mM, about 800 μM to about 5 mM, about 900 μM to about 1 mM, about 900 μM to about 2 mM, about 900 μM to about 3 mM, about 900 μM to about 4 mM, about 900 μM to about 5 mM, about 1 mM to about 2 mM, about 1 mM to about 3 mM, about 1 mM to about 4 mM, about 1 mM to about 5 mM, about 2 mM to about 3 mM, about 2 mM to about 4 mM, about 2 mM to about 5 mM, about 3 mM to about 4 mM, about 3 mM to about 5 mM, or about 4 mM to about 5 mM). In some embodiments, the cell suspension includes one or more ions (e.g., calcium ions or calcium chloride) at a concentration of about 0.1 mM.

In some embodiments, the addition of calcium chloride to the cell suspension enables the cells to seal the temporary membrane pores induced by viscoelastic stretching in less time compared to cells dispersed in a cell suspension that does not include calcium chloride or calcium ions.

In some embodiments, the cell suspension includes one or more antioxidants (e.g. Trolox, a vitamin E analog), poloxamer (e.g. Pluronic™ F-68, a membrane-stabilizing amphiphile that may help seal cell membrane pores), apoptosis inhibitors (e.g. Q-VD-Oph, a small molecule pan-Caspase inhibitor), glucose, a supplement used in cell culture cryorecovery medium, bovine serum albumin, fetal bovine serum, or any combination thereof. In some embodiments, the cell suspension includes a buffer (e.g., phosphate-buffered saline). In some embodiments, the cell suspension includes a cell culture medium (e.g., Roswell Park Memorial Institute (RPMI) 1640 Medium). In some embodiments, the cell suspension includes one or more components of a cell culture medium.

Also provided herein are methods of delivery of a substance to one or more particles. The method includes providing a substrate defining a micro-channel that connects a first chamber and a second chamber; and driving a particle suspension through the micro-channel, thereby: i) causing the one or more particles to be stretched along a direction of flow and ii) inducing a formation of one or more temporary pores in a membrane of the one or more particles. The particle suspension can include the one or more particles, a polymer, and the substance or cargo to be delivered. In some embodiments, the particle is not a cell (e.g., a mammalian, bacterial, or plant cell). In some embodiments, the methods described herein are used to deliver a substance to a particle (e.g., a particle that is not a cell). In some embodiments, the particle is a lipid nanoparticle or lipid microparticle (e.g., lipid particles having a diameter that is about 1 μm or greater). Non-limiting examples of a lipid nanoparticle or lipid microparticle include liposomes, vesicles, exosomes, or any combination thereof. In some embodiments, a particle is a cell (e.g., a nucleated cell or an enucleated cell), a synthetic nanoparticle, a nanoparticle composed of a naturally-derived molecule, a nanoparticle encapsulating a cargo composed of a synthetic or naturally-derived molecule, a nanoparticle surface-functionalized with a cargo composed of a synthetic or naturally-derived molecule, a microparticle composed of a naturally-derived molecule, a microparticle encapsulating a cargo composed of a synthetic or naturally-derived molecule, a microparticle surface-functionalized with a cargo composed of a synthetic or naturally-derived molecule. In some embodiments, the

In some embodiments, the particle can have a diameter of between about 100 nm to about 100 μm (e.g., about 100 nm to about 150 nm, about 100 nm to about 200 nm, about 100 nm to about 250 nm, about 100 nm to about 300 nm, about 100 nm to about 350 nm, about 100 nm to about 400 nm, about 100 nm to about 450 nm, about 100 nm to about 500 nm, about 100 nm to about 550 nm, about 100 nm to about 600 nm, about 100 nm to about 650 nm, about 100 nm to about 700 nm, about 100 nm to about 750 nm, about 100 nm to about 800 nm, about 100 nm to about 850 nm, about 100 nm to about 900 nm, about 100 nm to about 950 nm, about 100 nm to about 1,000 nm, about 150 nm to about 250 nm, about 200 nm and about 250 nm, about 250 nm to about 500 nm, about 500 nm and about 1,000 nm, about 700 nm and about 1,000 nm, about 800 nm and about 1,000 nm, about 1 μm to about 2 μm, about 1 μm to about 5 μm, about 1 μm to about 10 μm, about 1 μm to about 20 μm, about 1 μm to about 30 μm, about 1 μm to about 40 μm, about 1 μm to about 50 μm, about 1 μm to about 60 μm, about 1 μm to about 70 μm, about 1 μm to about 80 μm, about 1 μm to about 90 μm, about 1 μm to about 100 μm, about 10 μm to about 20 μm, about 10 μm to about 30 μm, about 10 μm to about 40 μm, about 10 μm to about 50 μm, about 10 μm to about 60 μm, about 10 μm to about 70 μm, about 10 μm to about 80 μm, about 10 μm to about 90 μm, about 10 μm to about 100 μm, about 20 μm to about 50 μm, about 20 μm to about 75 μm, about 20 μm to about 100 μm, about 50 μm to about 75 μm, about 50 μm to about 100 μm).

### Systems

Provided herein are systems for intracellular delivery of a substance or cargo to one or more cells. The systems include a microfluidic chip that is substantially similar in construction and function in several aspects to the microfluidic chip 100 discussed above. For example, the substrate defines a micro-channel in fluid communication with a first chamber and optionally in fluid communication with a second chamber and has a hydraulic diameter that is less than a hydraulic diameter of the first chamber and the optional second chamber. The system further includes a cell suspension including the cells, a polymer, and the substance or cargo, a pump configured to drive the cell suspension through the micro-channel, thereby: i) causing the one or more cells to be stretched along a direction of flow and ii) inducing a formation of one or more temporary pores in a membrane of the one or more cells, and a controller configured to control operation of the pump.

In some embodiments, the system include providing a substrate defining a micro-channel that connects a first chamber and a second chamber. In some embodiments, the system include providing a substrate defining a micro-channel extending between a first chamber and a second chamber. In some embodiments, the system includes a microfluidic chip or a device that includes a single chamber that is fluidically connected to a micro-channel defining an opening that is distal from the single chamber and that opens to an external environment. In some embodiments, the system includes a microfluidic chip or a device that includes a single chamber that is fluidically connected to a constriction defining an opening that is distal from the single chamber and that opens to an external environment.

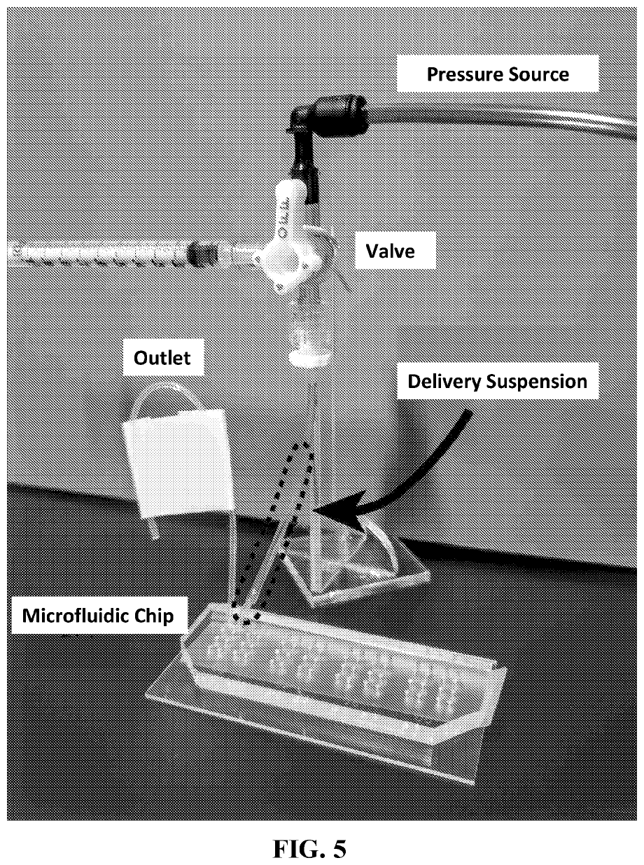

FIGS. 4A-4D are microscopy images of an exemplary microfluidic chip geometry. A cross-section of the channel is shown in FIG. 4D. In some embodiments, the micro-channel has a substantially square cross-section. The single microfluidic channel connects two tapered chambers having a height of about 125 microns and a width of about 1,500 microns. The micro-channel dimensions can be varied. In some embodiments, the microfluidic chip or device includes a single chamber that is fluidically connected to a micro-channel defining an opening that is distal from the single chamber and that opens to an external environment. In some embodiments, the microfluidic chip or device includes a single chamber that is fluidically connected to a constriction defining an opening that is distal from the single chamber and that opens to an external environment.

Referring back to FIG. 2, the micro-channel has a length l that extends between the first and second openings of the micro-channel and a hydraulic diameter d. In some embodiments, the length of the micro-channel is about 0 μm to about 500 μm (e.g., about 0 μm to about 1 μm, about 0 μm to about 2 μm, about 0 μm to about 3 μm, about 0 μm to about 4 μm, about 0 μm to about 5 μm, about 0 μm to about 6 μm, about 0 μm to about 7 μm, about 0 μm to about 8 μm, about 0 μm to about 9 μm, about 0 μm to about 10 μm, about 0 μm to about 15 μm, about 0 μm to about 20 μm, about 0 μm to about 25 μm, about 0 μm to about 30 μm, about 0 μm to about 35 μm, about 0 μm to about 40 μm, about 0 μm to about 45 μm, about 1 μm to about 5 μm, about 1 μm to about 15 μm, about 1 μm to about 25 μm, about 1 μm to about 50 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 10 μm to about 100 μm, about 10 μm to about 200 μm, about 10 μm to about 300 μm, about 10 μm to about 400 μm, about 10 μm to about 500 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 25 μm to about 50 μm, about 50 μm to about 100 μm, about 50 μm to about 200 μm, about 50 μm to about 300 μm, about 50 μm to about 400 μm, about 10 μm to about 500 μm, about 0 μm to about 50 μm, about 0 μm to about 100 μm, about 0 μm to about 150 μm, about 0 μm to about 200 μm, about 0 μm to about 250 μm, about 0 μm to about 300 μm, about 0 μm to about 350 μm, about 0 μm to about 400 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 50 μm to about 500 μm, about 100 μm to about 150 μm, about 100 μm to about 200 μm, about 100 μm to about 250 nm, about 100 nm to about 300 nm, about 100 nm to about 350 nm, or about 100 nm to about 400 nm, about 100 nm to about 500 nm, about 250 nm to about 500 nm). In some embodiments, the length of the micro-channel is about 100 nm. In some embodiments, the length of the micro-channel is about 0 nm. In some embodiments, a micro-channel having a length of about 0 nm is a pore.

In some embodiments, the hydraulic diameter d of the micro-channel is greater than a diameter of the cells subjected to or to be subjected to viscoelastic mechanoporation. In some embodiments, the hydraulic diameter d of the micro-channel is about twice as large as a diameter of the cells. In some embodiments, the diameter of the one or more cells ranges from about 4 nm to about 100 nm (e.g., about 4 nm to about 10 nm, about 4 nm to about 15 nm, about 4 nm to about 20 nm, about 4 nm to about 25 nm, about 4 nm to about 30 nm, about 4 nm to about 35 nm, about 4 nm to about 40 nm, about 4 nm to about 45 nm, about 4 nm to about 50 nm, about 4 nm to about 75 nm, or about 4 nm to about 100 nm). In some embodiments, the diameter of the one or more cells ranges is about 10 nm.

In some embodiments, the hydraulic diameter d of the micro-channel ranges from about 10 nm to about 100 nm (e.g., about 10 nm to about 30 nm, about 10 nm to about 40 nm, about 10 nm to about 50 nm, about 10 nm to about 60 nm, about 10 nm to about 70 nm, about 10 nm to about 80 nm, about 10 nm to about 90 nm, about 10 nm to about 95 nm, about 20 nm to about 30 nm, about 20 nm to about 40 nm, about 20 nm to about 50 nm, about 20 nm to about 60 nm, about 20 nm to about 70 nm, about 20 nm to about 80 nm, about 20 nm to about 90 nm, about 20 nm to about 100 nm, about 30 nm to about 40 nm, about 30 nm to about 50 nm, about 30 nm to about 60 nm, about 30 nm to about 70 nm, about 30 nm to about 80 nm, about 30 nm to about 90 nm, or about 30 nm to about 100 nm). In some embodiments, the hydraulic diameter d of the micro-channel is about 30 nm.

Still referring to FIG. 2, the first and second chambers are tapered and have a hydraulic diameter D, which gradually narrows into an opening of the micro-channel. In some embodiments, the hydraulic diameters D of the first and second chambers are about equivalent to each other. In some embodiments, the hydraulic diameter D of the first and second chambers ranges from about 300 nm to about 25,000 nm (e.g., about 300 nm to about 1,500 μm, about 300 μm to about 2,000 μm, about 300 μm to about 2,500 μm, about 300 μm to about 3000 μm, about 300 μm to about 5,000 μm, about 300 μm to about 10,000 μm, about 300 μm to about 15,000 μm, about 300 μm to about 20,000 μm, about 300 μm to about 25,000 μm, about 500 μm to about 1,500 μm, about 500 μm to about 2,000 μm, about 500 μm to about 2,500 μm, about 500 μm to about 3000 μm, about 500 μm to about 5,000 μm, about 500 μm to about 10,000 μm, about 500 μm to about 15,000 μm, about 500 μm to about 20,000 μm, about 500 μm to about 25,000 μm, about 1,000 μm to about 1,500 μm, about 1,000 μm to about 2,000 μm, about 1,000 μm to about 2,500 μm, about 1,000 μm to about 3000 μm, about 1,000 μm to about 5,000 μm, about 1,000 μm to about 10,000 μm, about 1,000 μm to about 15,000 μm, about 1,000 μm to about 20,000 μm, about 1,000 μm to about 25,000 μm, about 1,500 μm to about 2,000 μm, about 300 μm to about 2,500 μm, about 300 μm to about 3000 μm, about 300 μm to about 5,000 μm, about 300 μm to about 10,000 μm, about 300 μm to about 15,000 μm, about 300 μm to about 20,000 μm, or about 300 μm to about 25,000 μm). In some embodiments, the hydraulic diameter D of the first and second chambers is about 1,500 μm.

In some embodiments, a ratio of the smallest channel dimension (e.g., a hydraulic diameter) of the micro-channel to a diameter of a cell or particle ranges from about 2 to about 20 (e.g., about 2 to about 3, about 2 to about 4, about 2 to about 5, about 2 to about 6, about 2 to about 7, about 2 to about 8, about 2 to about 9, about 2 to about 10, about 2 to about 15, about 2 to about 20, about 5 to about 10, about 5 to about 15, about 5 to about 20, about 10 to about 15, about 10 to about 20, about 15 to about 20. In some embodiments, the ratio of the smallest channel dimension (e.g., a hydraulic diameter) of the micro-channel to a diameter of a cell or particle is about 2. In some embodiments, the ratio of the smallest channel dimension (e.g., a hydraulic diameter) of the micro-channel to a diameter of a cell or particle is about 3.

In some embodiments, a constriction ratio can be defined as the ratio of a cross-sectional area of a chamber (e.g., the first or second chambers) to a cross-sectional area of the micro-channel. In some embodiments, the constriction ratio ranges from about 10 to about 500 (e.g., about 10 to about 50, about 10 to about 60, about 10 to about 70, about 10 to about 80, about 10 to about 90, about 10 to about 100, about 10 to about 150, about 10 to about 200, about 10 to about 250, about 10 to about 300, about 10 to about 350, about 10 to about 400, about 10 to about 450, about 10 to about 495, about 40 to about 50, about 40 to about 60, about 40 to about 70, about 40 to about 80, about 40 to about 90, about 40 to about 100, about 40 to about 150, about 40 to about 200, about 40 to about 250, about 40 to about 300, about 40 to about 350, about 40 to about 400, about 40 to about 450, about 40 to about 500, about 50 to about 60, about 50 to about 70, about 50 to about 80, about 50 to about 90, about 50 to about 100, about 50 to about 150, about 50 to about 200, about 50 to about 250, about 50 to about 300, about 50 to about 350, about 50 to about 400, about 50 to about 450, or about 50 to about 500). In some embodiments, the constriction ratio is about 50. In some embodiments, the constriction ratio is about 150.

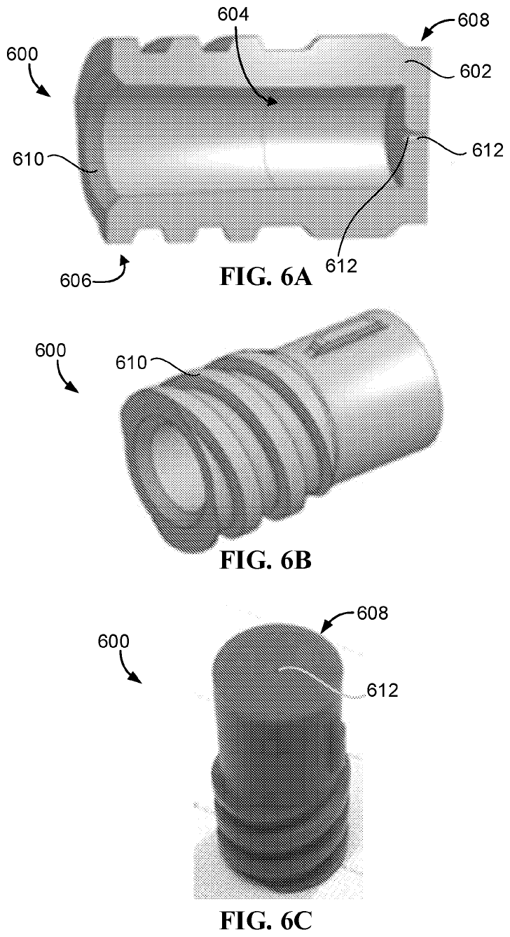

Referring to FIG. 5, the microfluidic system includes a pump that includes a regulated pressure line, which is in fluid connection with an inlet of the microfluidic chip. The pump is configured to drive the cell suspension through the micro-channel by applying a driving pressure. In some embodiments, the pump is configured to drive the cell suspension from the first chamber, through the micro-channel, into the second chamber, and further through an outlet. The microfluidic system further includes a valve in fluid communication with the inlet of the microfluidic chip and micro-channel and with the pressure line. The valve is configured to selectively control the driving pressure. For example, a user may actuate the valve to place it in an “on” position, thereby allowing the compressed air to flow through a tubing that is in fluid communication with the microfluidic chip. In another example, a user may actuate the valve to place it in an “off” position, thereby preventing the compressed air from flowing through the tubing and preventing a cell suspension to be driven into a component of the microfluidic chip (e.g., the micro-channel or one of the chambers).

The system includes a controller configured to control or regulate the volumetric flow rates at which the pump operates. In some embodiments, the system can include one or more sensors configured to detect a change in a flow rate and/or a change in a pressure of the system. In some embodiments, the controller can be configured to receive sensor data indicative of a change in a flow rate and/or a pressure, process the received sensor data, and optionally transmit the processed sensor data to an external source (e.g., a computing device). In some embodiments, the computing device is a remote computing device. In some embodiments, the controller is configured to transmit the processed sensor data over a wired or wireless network to an external source (e.g., a remote computing device). In some embodiments, the controller is configured to reduce a volumetric flow rate at which the pump is operating or to stop the operation pump altogether if the processed sensor data indicates the flow rate has decreased or a pressure has increased (e.g., due to a blockage). In some embodiments, the controller is configured to reduce a volumetric flow rate at which the pump is operating or to stop the operation pump altogether if the processed sensor data indicates the flow rate has increased or a pressure has decreased (e.g., due to a leakage).

In some embodiments, the controller is configured to control the pump to drive the cell suspension through the micro-channel at a volumetric flow rate ranging from about 0.1 ml/minute to about 20 ml/minute (e.g., about 0.1 ml/min to about 0.5 ml/min, about 0.5 ml/min to about 1 ml/min, about 0.1 ml/min to about 1 ml/min, about 0.1 ml/min to about 3 ml/min, about 0.1 ml/min to about 4 ml/min, about 0.1 ml/min to about 5 ml/min, about 0.1 ml/min to about 6 ml/min, about 0.1 ml/min to about 7 ml/min, about 0.1 ml/min to about 8 ml/min, about 0.1 ml/min to about 9 ml/min, about 0.1 ml/min to about 10 ml/min, about 0.1 ml/min to about 15 ml/min, or about 0.1 ml/min to about 20 ml/min).

In some embodiments, the controller is configured to control the pump to drive the cell suspension through the micro-channel at a volumetric flow rate resulting in a maximum Reynolds number of about 10 to about 100 (e.g., about 10 to about 20, about 10 to about 30, about 10 to about 40, about 10 to about 50, about 10 to about 60, about 10 to about 70, about 10 to about 80, about 10 to about 90, about 10 to about 100, about 25 to about 50, about 25 to about 75, about 25 to about 100, about 50 to about 75, or about 50 to about 100).

A system may be substantially similar in construction and function in several aspects to the systems discussed above, but can include an alternative microfluidic chip instead of the microfluidic chip 100. In some embodiments, the microfluidic chip may have alternative microfluidic channel configurations. For example, the microfluidic channel may have a spiral configuration. Such spiral configurations can allow a user to control the acceleration and/or position of the particles within the microfluidic channel to achieve, for example, an equal distribution of stress on each cell such that each cell is exposed to the same composition of impinging stresses and strains as well as the same duration of those stresses and strains.

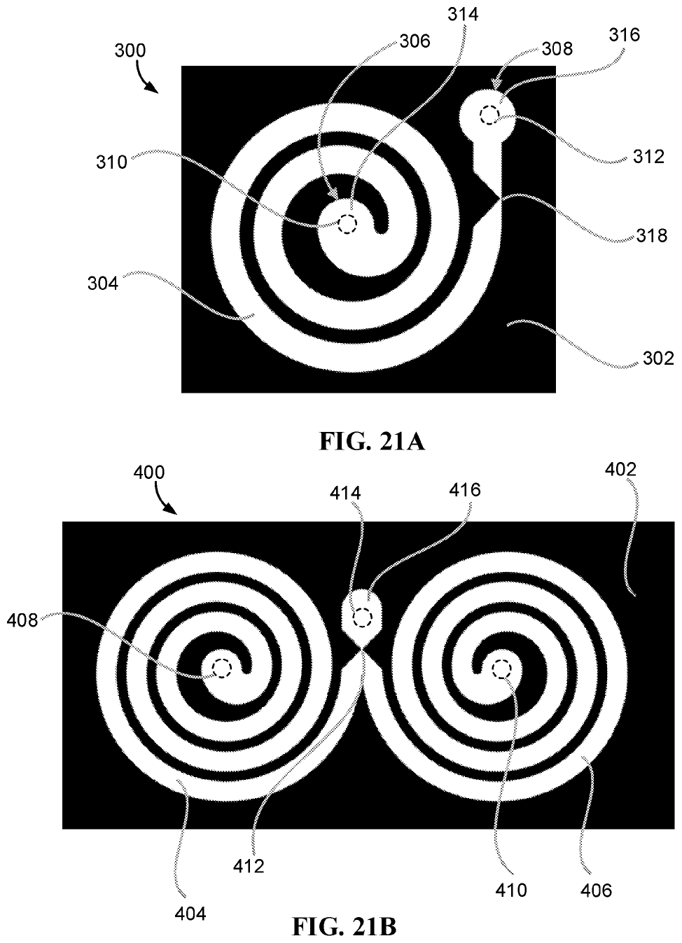

FIG. 21A is a schematic illustrating a top view of an outline of an exemplary microfluidic chip 300. The microfluidic chip 300 includes a substrate 302 (black area) defining a channel 304 (outlined by the white area) having a spiral geometry. The channel 304 extends from a first end 306 to a second end 308 in a generally spiral path. The first end 306 and the second end 308 have an inlet pad 314 and an outlet pad 316, respectively, that are generally circular in shape. The inlet pad 314 defines an inlet 310. The inlet 310 is located at the first end 306, at or near a center of the spiral path, and at or near a center of the inlet pad 314. The inlet 310 is configured to receive any of the cell suspensions described elsewhere herein.

In some embodiments, the inlet pad 314 has a diameter that ranges from about 0.5 mm to about 10 mm (e.g., about 0.5 mm to about 1 mm, about 0.5 mm to about 1.5 mm, about 0.5 mm to about 2 mm, about 0.5 mm to about 3 mm, about 0.5 mm to about 5 mm, about 1 mm to about 1.5 mm, about 1 mm to about 2 mm, about 1 mm to about 3 mm, about 1 mm to about 4 mm, about 1 mm to about 5 mm, about 1 mm to about 10 mm, about 2 mm to about 3 mm, about 2 mm to about 4 mm, about 2 mm to about 5 mm, about 2 mm to about 10 mm, or more). In some embodiments, the inlet pad 314 has a diameter of about 2 mm.

In some embodiments, the inlet 310 is in fluid connection with a chamber. In some embodiments, the inlet 310 is in fluid connection with a second channel. In some embodiments, the inlet 310 is located at any suitable location (e.g., off-center) with respect to the spiral path of the channel. In some embodiments, the inlet 310 is defined by the inlet pad 314 at any suitable location within an area of the inlet pad 314 (e.g., off-center or near a wall of the inlet pad 314).

The outlet pad 316 defines an outlet 312. The outlet 312 is located at the second end 308, near a center of the outlet pad 316. In some embodiments, the cell or particle suspension is collected at or near the outlet 312. In some embodiments, a cell or a particle from the cell suspension or the particle suspension, respectively, is collected, detected, counted, or otherwise analyzed at or near the outlet 312. In some embodiments, the outlet 312 is in fluid connection with a chamber. In some embodiments, the outlet 312 is in fluid connection with a second channel.

In some embodiments, the outlet pad 316 has a diameter that ranges from about 0.5 mm to about 10 mm (e.g., about 0.5 mm to about 1 mm, about 0.5 mm to about 1.5 mm, about 0.5 mm to about 2 mm, about 0.5 mm to about 3 mm, about 0.5 mm to about 5 mm, about 1 mm to about 1.5 mm, about 1 mm to about 2 mm, about 1 mm to about 3 mm, about 1 mm to about 4 mm, about 1 mm to about 5 mm, about 1 mm to about 10 mm, about 2 mm to about 3 mm, about 2 mm to about 4 mm, about 2 mm to about 5 mm, about 2 mm to about 10 mm, or more). In some embodiments, the outlet pad 316 has a diameter of about 2 mm.

In some embodiments, the outlet 312 is in fluid connection with a chamber. In some embodiments, the outlet 312 is in fluid connection with a second channel. In some embodiments, the outlet 312 is located at any suitable location with respect to the spiral path of the channel and/or the inlet 310 (e.g., distal from the inlet 310). In some embodiments, the outlet 312 is defined by the outlet pad 316 at any suitable location within an area of the outlet pad 316 (e.g., off-center or near a wall of the outlet pad 316).

The channel 304 has a width and a hydraulic diameter that is about the same throughout its length until reaching a constriction 318 where the width and the hydraulic diameter are reduced. The channel 304 narrows to the constriction 318 near the distal end 308. In some embodiments, the channel 304 has a width and a hydraulic diameter of about 1 mm that is about the same throughout its length until reaching the constriction 318 where the width and the hydraulic diameter are reduced to about 50 μm. In some embodiments, the channel 304 has a width ranging from about 500 μm to about 2,000 μm (e.g., about 500 μm to about 750 μm, about 750 μm to about 1,000 μm, about 1,000 μm to about 1,250 μm, about 1,000 μm to about 1,500 μm, about 1,500 μm to about 1,750 μm, about 1,750 μm to about 2,000 μm, about 500 μm to about 1,000 μm, about 500 μm to about 1,500 μm, about 500 μm to about 2000 μm, about 1,000 μm to about 1,750 μm, or about 1,000 μm to about 2,000 μm). In some embodiments, the channel 304 has a width of about 1,000 μm.

In some embodiments, the channel 304 has a hydraulic diameter ranging from about 500 μm to about 2,000 μm (e.g., about 500 μm to about 750 μm, about 750 μm to about 1,000 μm, about 1,000 μm to about 1,250 μm, about 1,000 μm to about 1,500 μm, about 1,500 μm to about 1,750 μm, about 1,750 μm to about 2,000 μm, about 500 μm to about 1,000 μm, about 500 μm to about 1,500 μm, about 500 μm to about 2000 μm, about 1,000 μm to about 1,750 μm, or about 1,000 μm to about 2,000 μm). In some embodiments, the channel 304 has a hydraulic diameter of about 1,000 μm.

The generally spiral path of the channel 304 extends at a length ranging from about 5 mm to about 20 mm (e.g., about 5 mm to about 6 mm, about 5 mm to about 7 mm, about 5 mm to about 8 mm, about 5 mm to about 9 mm, about 5 mm to about 10 mm, about 5 mm to about 11 mm, about 5 mm to about 12 mm, about 5 mm to about 13 mm, about 5 mm to about 14 mm, about 5 mm to about 15 mm, about 5 mm to about 16 mm, about 5 mm to about 17 mm, about 5 mm to about 18 mm, about 5 mm to about 19 mm, about 10 mm to about 15 mm, about 10 mm to about 20 mm, or about 15 mm to about 20 mm), as measured from the first end 306 or the inlet 310 to the constriction 318.

In some embodiments, the constriction 318 has a width ranging from about 10 μm to about 100 μm (e.g., about 10 μm to about 30 μm, about 10 μm to about 40 μm, about 10 μm to about 50 μm, about 10 μm to about 60 μm, about 10 μm to about 70 μm, about 10 μm to about 80 μm, about 10 μm to about 90 μm, about 10 μm to about 95 μm, about 20 μm to about 30 μm, about 20 μm to about 40 μm, about 20 μm to about 50 μm, about 20 μm to about 60 μm, about 20 μm to about 70 μm, about 20 μm to about 80 μm, about 20 μm to about 90 μm, about 20 μm to about 100 μm, about 30 μm to about 40 μm, about 30 μm to about 50 μm, about 30 μm to about 60 μm, about 30 μm to about 70 μm, about 30 μm to about 80 μm, about 30 μm to about 90 μm, or about 30 μm to about 100 μm). In some embodiments, the constriction 318 has a width of about 50 μm.

In some embodiments, the constriction 318 has a hydraulic diameter ranging from about 10 μm to about 100 μm (e.g., about 10 μm to about 30 μm, about 10 μm to about 40 μm, about 10 μm to about 50 μm, about 10 μm to about 60 μm, about 10 μm to about 70 μm, about 10 μm to about 80 μm, about 10 μm to about 90 μm, about 10 μm to about 95 μm, about 20 μm to about 30 μm, about 20 μm to about 40 μm, about 20 μm to about 50 μm, about 20 μm to about 60 μm, about 20 μm to about 70 μm, about 20 μm to about 80 μm, about 20 μm to about 90 μm, about 20 μm to about 100 μm, about 30 μm to about 40 μm, about 30 μm to about 50 μm, about 30 μm to about 60 μm, about 30 μm to about 70 μm, about 30 μm to about 80 μm, about 30 μm to about 90 μm, or about 30 μm to about 100 μm). In some embodiments, the constriction 318 has a hydraulic diameter of about 50 μm.

In some embodiments, the constriction 318 has a length ranging from about 0 μm to about 400 μm (e.g., about 0 μm to about 1 μm, about 0 μm to about 2 μm, about 0 μm to about 3 μm, about 0 μm to about 4 μm, about 0 μm to about 5 μm, about 0 μm to about 6 μm, about 0 μm to about 7 μm, about 0 μm to about 8 μm, about 0 μm to about 9 μm, about 0 μm to about 10 μm, about 0 μm to about 15 μm, about 0 μm to about 20 μm, about 0 μm to about 25 μm, about 0 μm to about 30 μm, about 0 μm to about 35 μm, about 0 μm to about 40 μm, about 0 μm to about 45 μm, about 1 μm to about 5 μm, about 1 μm to about 15 μm, about 1 μm to about 25 μm, about 1 μm to about 50 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 25 μm to about 50 μm, about 0 μm to about 50 μm, about 0 μm to about 100 μm, about 0 μm to about 150 μm, about 0 μm to about 200 μm, about 0 μm to about 250 μm, about 0 μm to about 300 μm, about 0 μm to about 350 μm, about 0 μm to about 400 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 100 μm to about 150 μm, about 100 μm to about 200 μm, about 100 μm to about 250 μm, about 100 μm to about 300 μm, about 100 μm to about 350 μm, or about 100 μm to about 400 μm). In some embodiments, the constriction 318 has a length of about 5 μm.

In some embodiments, a constriction ratio can be defined as the ratio of a cross-sectional area of the channel 304 to a cross-sectional area of the constriction 318. In some embodiments, the constriction ratio (e.g., the ratio of a cross-sectional area of the channel 304 to a cross-sectional area of the constriction 318) ranges from about 10 to about 500 (e.g., about 10 to about 50, about 10 to about 60, about 10 to about 70, about 10 to about 80, about 10 to about 90, about 10 to about 100, about 10 to about 150, about 10 to about 200, about 10 to about 250, about 10 to about 300, about 10 to about 350, about 10 to about 400, about 10 to about 450, about 10 to about 495, about 40 to about 50, about 40 to about 60, about 40 to about 70, about 40 to about 80, about 40 to about 90, about 40 to about 100, about 40 to about 150, about 40 to about 200, about 40 to about 250, about 40 to about 300, about 40 to about 350, about 40 to about 400, about 40 to about 450, about 40 to about 500, about 50 to about 60, about 50 to about 70, about 50 to about 80, about 50 to about 90, about 50 to about 100, about 50 to about 150, about 50 to about 200, about 50 to about 250, about 50 to about 300, about 50 to about 350, about 50 to about 400, about 50 to about 450, or about 50 to about 500). In some embodiments, the constriction ratio (e.g., the ratio of a cross-sectional area of the channel 304 to a cross-sectional area of the constriction 318) is about 50. In some embodiments, the constriction ratio (e.g., the ratio of a cross-sectional area of the channel 304 to a cross-sectional area of the constriction 318) is about 150.

In some embodiments, the channel 304 has a cross-section that is substantially square. In some embodiments, the channel 304 has a cross-section that is substantially quadrilateral (e.g., rectangular, parallelogram, rhomboid, kite, trapezoid) cross-section. In some embodiments, the channel 304 has a cross-section that is substantially circular, oval, cylindrical, or semi-cylindrical. In some embodiments, the channel 304 has a cross-section that is substantially curved, triangular, hexagonal, or any other suitable shape. In some embodiments, the channel 304 may alternatively have a combination of one or more types or shapes of cross-sections. For example, in some embodiments, the channel 304 includes a portion that is substantially rectangular in cross-section and a portion that is substantially circular or oval in-cross section.

A microfluidic chip may be substantially similar in construction and function in several aspects to the microfluidic chip 300 discussed above, but can include, alternatively, one or more channels instead of a single channel 304. In some embodiments, the microfluidic chip may have two channels. For example, the microfluidic chip may have a first spiral channel and a second spiral channel in fluid communication with each other.

FIG. 21B is a illustrates an example of a microfluidic chip 400 including a substrate 402 (black area) defining a first channel 404 and a second channel 406 (both outlined by the white area), each having a spiral geometry. The first channel 404 includes a first inlet 408, and the second channel 406 includes a second inlet 410. The first and second channels 404, 406 are in fluid communication with each other and converge at a constriction 412. An outlet 414 is defined by an outlet pad 416 located distally from the constriction 412.

In some embodiments, the first and second channels 404, 406 can include one or more dimensions, shapes, arrangements, and/or configurations and perform functions that are the same from the ones discussed above with respect to the channel 304. In some embodiments, the first and second inlets 408, 410 can include one or more dimensions, shapes, arrangements, and/or configurations and perform functions that are the same from the ones discussed above with respect to the inlet 310. The constriction 416 can include one or more dimensions, shapes, arrangements, and/or configurations and perform functions that are the same from the ones discussed above with respect to the constriction 318.

In some embodiments, the first and second channels 404, 406 are configured to receive a same cell or particle suspension. In some embodiments, the first and second channels 404, 406 are configured to receive a different cell or particle suspension. For example, the first channel 404 is configured to receive a first cell or particle suspension, and the second channel 406 is configured to receive a second cell or particle suspension.

A system may be substantially similar in construction and function in several aspects to the systems discussed above, but can include an alternative microfluidic device instead of the microfluidic chip 100. In some embodiments, the system may have an alternative microfluidic device and alternative configurations. For example, the system may have a microfluidic device configured to reversibly connect to a liquid handling system or a microwell plate. Such microfluidic devices can allow a user to directly couple the microfluidic device to a liquid handling system or microwell plate without the use of additional tubing or connectors, thereby simplifying and streamlining the cell stretching and mechanoporation process.

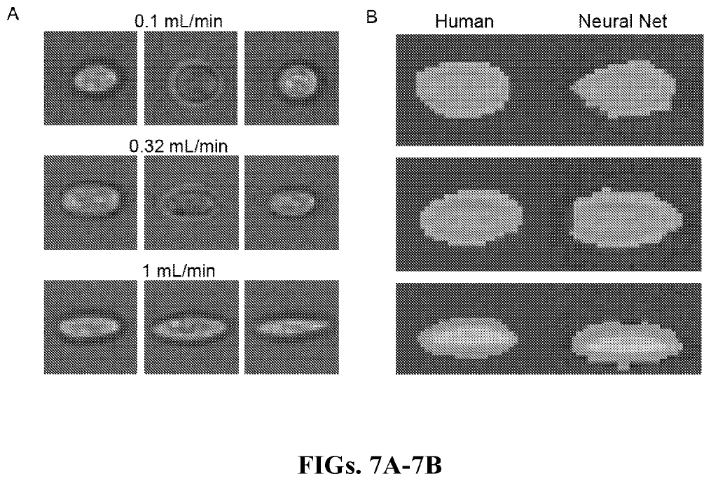

FIGS. 6A-6C show a microfluidic device 600 including a substrate 602 (e.g., a wall defining a chamber 604. The substrate 602 can be composed of plastic or other suitable material. The chamber 604 has a cylindrical shape having a semi-cylindrical cross-section, as shown in FIG. 6A. The chamber 604 has a cylindrical body extending from a first end 606 to a second end 608. The first end 606 defines an inlet 610 that is circular in shape and is configured to fluidically connect to another device (e.g., a syringe, a liquid handling system, or a microplate). The second end 608 defines an outlet 612, as shown in FIG. 6C. In some embodiments, a cell or particle suspension is configured to be collected at or near the outlet 612. The chamber 604 has a hydraulic diameter that is about the same throughout a length of the chamber 604 until reaching a constriction 606 where the hydraulic diameter is gradually reduced.

In some embodiments, the hydraulic diameter of the chamber 604 ranges from about 200 μm to about 50,000 μm (e.g., about 200 μm to about 300 μm, about 200 μm to about 400 μm, about 200 μm to about 500 μm, about 200 μm to about 1,000 μm, about 200 μm to about 5,000 μm, about 200 μm to about 10,000 μm, about 200 μm to about 15,000 μm, about 200 μm to about 25,000 μm, about 200 μm to about 50,000 μm, about 300 μm to about 1,500 μm, about 300 μm to about 2,000 μm, about 300 μm to about 2,500 μm, about 300 μm to about 3000 μm, about 300 μm to about 5,000 μm, about 300 μm to about 10,000 μm, about 300 μm to about 15,000 μm, about 300 μm to about 20,000 μm, about 300 μm to about 25,000 μm, about 500 μm to about 1,500 μm, about 500 μm to about 2,000 μm, about 500 μm to about 2,500 μm, about 500 μm to about 3000 μm, about 500 μm to about 5,000 μm, about 500 μm to about 10,000 μm, about 500 μm to about 15,000 μm, about 500 μm to about 20,000 μm, about 500 μm to about 25,000 μm, about 1,000 μm to about 1,500 μm, about 1,000 μm to about 2,000 μm, about 1,000 μM to about 2,500 μm, about 1,000 μm to about 3000 μm, about 1,000 μm to about 5,000 μm, about 1,000 μm to about 10,000 μm, about 1,000 μm to about 15,000 μm, about 1,000 μm to about 20,000 μm, about 1,000 μm to about 25,000 μm, about 1,500 μm to about 2,000 μm, about 300 μm to about 2,500 μm, about 300 μm to about 3000 μm, about 300 μm to about 5,000 μm, about 300 μm to about 10,000 μm, about 300 μm to about 15,000 μm, about 300 μm to about 20,000 μm, or about 300 μm to about 25,000 μm).

In some embodiments, the constriction 606 has a hydraulic diameter ranging from about 50 μm to about 1,000 μm (e.g., about 50 μm to about 60 μm, about 50 μm to about 70 μm, about 50 μm to about 80 μm, about 50 μm to about 90 μm, about 50 μm to about 100 μm, about 50 μm to about 125 μm, about 50 μm to about 150 μm, about 50 μm to about 175 μm, about 50 μm to about 200 μm, about 50 μm to about 250 μm, about 50 μm to about 300 μm, about 50 μm to about 350 μm, about 50 μm to about 400 μm, about 50 μm to about 450 μm, about 50 μm to about 500 μm, about 50 μm to about 550 μm, about 50 μm to about 600 μm, about 50 μm to about 650 μm, about 50 μm to about 700 μm, about 50 μm to about 750 μm, about 50 μm to about 800 μm, about 50 μm to about 850 μm, about 50 μm to about 900 μm, about 50 μm to about 950 μm, about 100 μm to about 200 μm, about 100 μm to about 250 μm, about 100 μm to about 500 μm, about 100 μm to about 1,000 μm, about 150 μm to about 500 μm, about 150 μm to about 1,000 μm, about 200 μm to about 250 μm, about 200 μm to about 500 μm, about 200 μm to about 1,000 μm, about 250 μm to about 500 μm, about 250 μm to about 1,000 μm, about 500 μm to about 1,000 μm, or about 750 μm to about 1,000 μm). In some embodiments, the constriction 606 has a hydraulic diameter of about 50 μm. In some embodiments, the constriction 606 has a hydraulic diameter of about 100 μm. In some embodiments, the constriction 606 has a hydraulic diameter of about 150 μm. In some embodiments, the constriction 606 has a hydraulic diameter of about 200 μm.

In some embodiments, the constriction 606 has a length ranging from about 0 μm to about 400 μm (e.g., about 0 μm to about 1 μm, about 0 μm to about 2 μm, about 0 μm to about 3 μm, about 0 μm to about 4 μm, about 0 μm to about 5 μm, about 0 μm to about 6 μm, about 0 μm to about 7 μm, about 0 μm to about 8 μm, about 0 μm to about 9 μm, about 0 μm to about 10 μm, about 0 μm to about 15 μm, about 0 μm to about 20 μm, about 0 μm to about 25 μm, about 0 μm to about 30 μm, about 0 μm to about 35 μm, about 0 μm to about 40 μm, about 0 μm to about 45 μm, about 1 μm to about 5 μm, about 1 μm to about 15 μm, about 1 μm to about 25 μm, about 1 μm to about 50 μm, about 10 μm to about 15 μm, about 10 μm to about 20 μm, about 10 μm to about 25 μm, about 10 μm to about 30 μm, about 10 μm to about 35 μm, about 10 μm to about 40 μm, about 10 μm to about 45 μm, about 10 μm to about 50 μm, about 15 μm to about 20 μm, about 15 μm to about 25 μm, about 15 μm to about 30 μm, about 15 μm to about 35 μm, about 15 μm to about 40 μm, about 15 μm to about 45 μm, about 15 μm to about 50 μm, about 25 μm to about 50 μm, about 0 μm to about 50 μm, about 0 μm to about 100 μm, about 0 μm to about 150 μm, about 0 μm to about 200 μm, about 0 μm to about 250 μm, about 0 μm to about 300 μm, about 0 μm to about 350 μm, about 0 μm to about 400 μm, about 50 μm to about 100 μm, about 50 μm to about 150 μm, about 50 μm to about 200 μm, about 50 μm to about 250 μm, about 50 μm to about 300 μm, about 50 μm to about 350 μm, about 50 μm to about 400 μm, about 100 μm to about 150 μm, about 100 μm to about 200 μm, about 100 μm to about 250 μm, about 100 μm to about 300 μm, about 100 μm to about 350 μm, or about 100 μm to about 400 μm). In some embodiments, the constriction 606 has a length of about 5 μm.

In some embodiments, a constriction ratio can be defined as the ratio of a cross-sectional area of the chamber 604 to a cross-sectional area of the constriction 606. In some embodiments, the constriction ratio (e.g., the ratio of a cross-sectional area of the channel 604 to a cross-sectional area of the constriction 606) ranges from about 10 to about 500 (e.g., about 10 to about 50, about 10 to about 60, about 10 to about 70, about 10 to about 80, about 10 to about 90, about 10 to about 100, about 10 to about 150, about 10 to about 200, about 10 to about 250, about 10 to about 300, about 10 to about 350, about 10 to about 400, about 10 to about 450, about 10 to about 495, about 40 to about 50, about 40 to about 60, about 40 to about 70, about 40 to about 80, about 40 to about 90, about 40 to about 100, about 40 to about 150, about 40 to about 200, about 40 to about 250, about 40 to about 300, about 40 to about 350, about 40 to about 400, about 40 to about 450, about 40 to about 500, about 50 to about 60, about 50 to about 70, about 50 to about 80, about 50 to about 90, about 50 to about 100, about 50 to about 150, about 50 to about 200, about 50 to about 250, about 50 to about 300, about 50 to about 350, about 50 to about 400, about 50 to about 450, or about 50 to about 500). In some embodiments, the constriction ratio (e.g., the ratio of a cross-sectional area of the channel 604 to a cross-sectional area of the constriction 606) is about 50. In some embodiments, the constriction ratio (e.g., the ratio of a cross-sectional area of the channel 604 to a cross-sectional area of the constriction 606) is about 150.

Referring to FIG. 6B, the microfluidic device 600 includes a locking connector 614 (e.g., a Luer taper connector) at the first end 606. The locking connector 614 is configured to reversibly and fluidically connect the microfluidic device 600 to another device (e.g., a syringe, a liquid handling system, or a microplate). In some embodiments, in operation, the locking connector 614 is reversibly and fluidically connected to a syringe that transfers a cell or particle suspension to the microfluidic device 600 via the inlet 610. In some embodiments, the outlet 612 is fluidically connected to a second chamber. In some embodiments, the outlet 612 is fluidically connected to a collection chamber. In some embodiments, the outlet 612 is fluidically connected to a collection tube.

In some embodiments, the inlet 610 can include one or more dimensions, shapes, arrangements, and/or configurations and perform functions that are the same from the ones discussed above with respect to any of the inlets described elsewhere herein. In some embodiments, the outlet 612 can include one or more dimensions, shapes, arrangements, and/or configurations and perform functions that are the same from the ones discussed above with respect to any of the inlets described elsewhere herein.

While the above-discussed microfluidic chip 100 and micro-channel 108 have been described and illustrated with respect to certain dimensions, shapes, arrangements, configurations, material formulations, and methods, in some embodiments, a microfluidic chip 100 or micro-channel 108 that is otherwise substantially similar in construction and function to the microfluidic chip 100 or micro-channel 108 may include one or more dimensions, shapes, arrangements, configurations, and/or materials formulations that are different from the ones discussed above or may be used with respect to methods that are modified as compared to the methods described above. For example, while the micro-channel 108 has been described and illustrated as having a substantially square cross-section, in some embodiments, a micro-channel that is otherwise substantially similar in construction and function to the micro-channel 108 may alternatively have a substantially quadrilateral (e.g., rectangular, parallelogram, rhomboid, kite, trapezoid) cross-section, a substantially circular or oval cross-section, cylindrical, semi-cylindrical, having two first walls and two second walls. In some embodiments, the first walls define a width of the fluidic channel, and, in some embodiments, the second walls define a height of the fluidic channel. In some embodiments, the micro-channel may alternatively have a substantially circular or oval cross-section. In some embodiments, the micro-channel having a substantially circular or oval cross-section is defined, in part, by an internal diameter and/or radius. In some embodiments, the micro-channel may alternatively have a substantially curved, triangular, hexagonal, or any other suitable cross-section. In some embodiments, the micro-channel may alternatively have a combination of one or more types or shapes of cross-sections. For example, in some embodiments, the micro-channel includes a portion that is substantially rectangular in cross-section and a portion that is substantially circular or oval in-cross section.

In some embodiments, an inner inlet and an outer inlet can be fluidly coupled to a spiral fluidic channel that is arranged in a plurality of loops. In some embodiments, a fluidic channel can be of a substantially quadrilateral (e.g., rectangular, parallelogram, rhomboid, kite, trapezoid) cross section, having two first walls and two second walls. In some embodiments, the first walls define a width of the fluidic channel, and (in some embodiments) the second walls define a height of the fluidic channel. In some embodiments, a fluidic channel is substantially circular or oval in cross section. In some embodiments, a fluidic channel includes a portion that is substantially rectangular in cross section and a portion that is substantially circular or oval in cross section. In some embodiments, a fluidic channel having a substantially circular or oval cross section is defined, in part, by an internal diameter and/or radius.

In another implementation, while the system has been described and illustrated as having a single micro-channel, in some embodiments, a system that is otherwise substantially similar in construction and function to the system discussed above may alternatively further include a second or more micro-channels connecting the first or 25 second chambers to a third or more chambers in parallel. For example, in some embodiments, a system that is otherwise substantially similar in construction and function to the system discussed above may alternatively further include about 10 micro-channels to about 20 micro-channels. In some embodiments, a microfluidic system having two or more micro-channels (e.g., less than about 20 micro-channels) can achieve viscoelastic mechanoporation and intracellular delivery of a cargo at a rate of about 1 billion cells per minute.

In another implementation, while the system has been described and illustrated as having a single micro-channel, in some embodiments, a system that is otherwise substantially similar in construction and function to the system discussed above may alternatively include a channel that is not a microfluidic channel (e.g., the channel has dimensions that are larger than the dimensions of a microfluidic channel). For example, in some embodiments, a system that is otherwise substantially similar in construction and function to the system discussed above may alternatively include a channel that has a cross-sectional area that is about 1 or 2 orders of magnitude larger (e.g., about 10 times or about 100 times larger) than any of the cross-sectional areas of the micro-channel disclosed elsewhere herein.

In another implementation, while the system has been described and illustrated as including a pump, in some embodiments, a system that is otherwise substantially similar in construction and function to the system discussed above may alternatively include a syringe. For example, in some embodiments, a system that is otherwise substantially similar in construction and function to the system discussed above may alternatively include a syringe that is in fluid connection with an inlet of any of the microfluidic chips or devices disclosed herein and further operatively connected to a syringe drive or a syringe pump.

### EXAMPLES

Certain embodiments of the present disclosure are further described in the following examples, which do not limit the scope of any embodiments described in the claims.

### Example 1. Fabrication of Micro-Channel and Preparation of Cell Suspension

To evaluate whether cells would be stretched and permeabilized by a viscoelastic extensional flow, a microfluidic constriction and a biocompatible viscoelastic solution were devised. Microfluidic constrictions were fabricated from micropatterned polydimethylsiloxane (PDMS) bonded to glass. The geometry included a single micro-channel 100 μm in length, with a cross-section of 35 nm by 55 nm, that connected two tapered chambers approximately 125 nm in height and 1,500 nm in width (FIGS. 4A-4D). A biocompatible and biodegradable viscoelastic solution was formulated with hyaluronic acid (HA), a high molecular weight linear glycosaminoglycan, in concentrations ranging from about 0.006% to about 0.4% w/v. Except where stated otherwise, a delivery solution of 0.2% w/v HA in phosphate buffered saline (PBS) with 0.1 mM CaCl₂) was used. Jurkat cells were dispersed in the delivery solution at concentrations of about 1×10⁶ to about 100×10⁶ per ml. In a typical experiment, 50 μl of the cell suspension was pneumatically aspirated into a short length of tubing, which was then press-fit into the inlet of the PDMS-glass device. The sample was completely pumped through the chip by releasing a valve and applying a regulated driving pressure of about 0.5 to about 6 bar (FIG. 5). The cell suspension was collected and immediately diluted 10-fold in prewarmed cell culture medium (Roswell Park Memorial Institute (RPMI)-1640 medium supplemented with 10% fetal bovine serum and 100 U/mL penicillin-streptomycin). Cells were incubated for about 5 to about 30 minutes at about 37° C. before washing by centrifugation and resuspension in pre-warmed culture medium. Control “unstretched” cells were incubated in the delivery solution containing the cargo molecule but were not pumped through the device. Cell viability and delivery efficiency were assessed after overnight incubation except where indicated otherwise. Cell viability was assessed by propidium iodide (PI) exclusion. Fluorescence intensity of fluorescein isothiocyanate (FITC) and PI were assessed by imaging flow cytometry.

### Example 2. Assessment of Cell Stretching, Mechanoporation, and Intracellular Delivery in Jurkat Cells

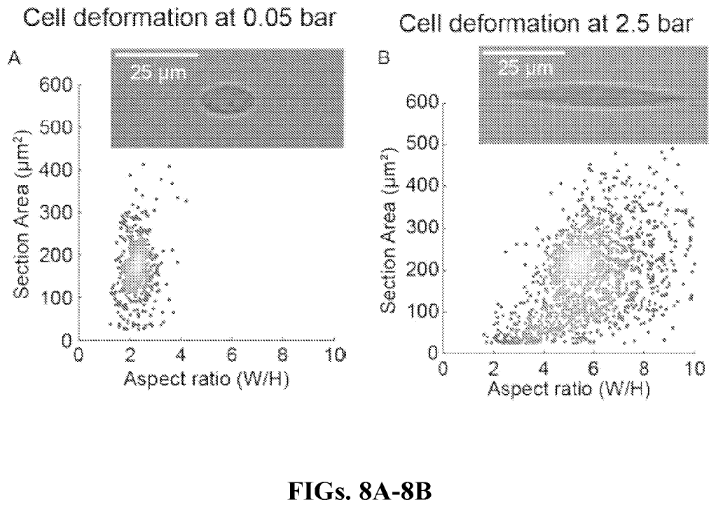

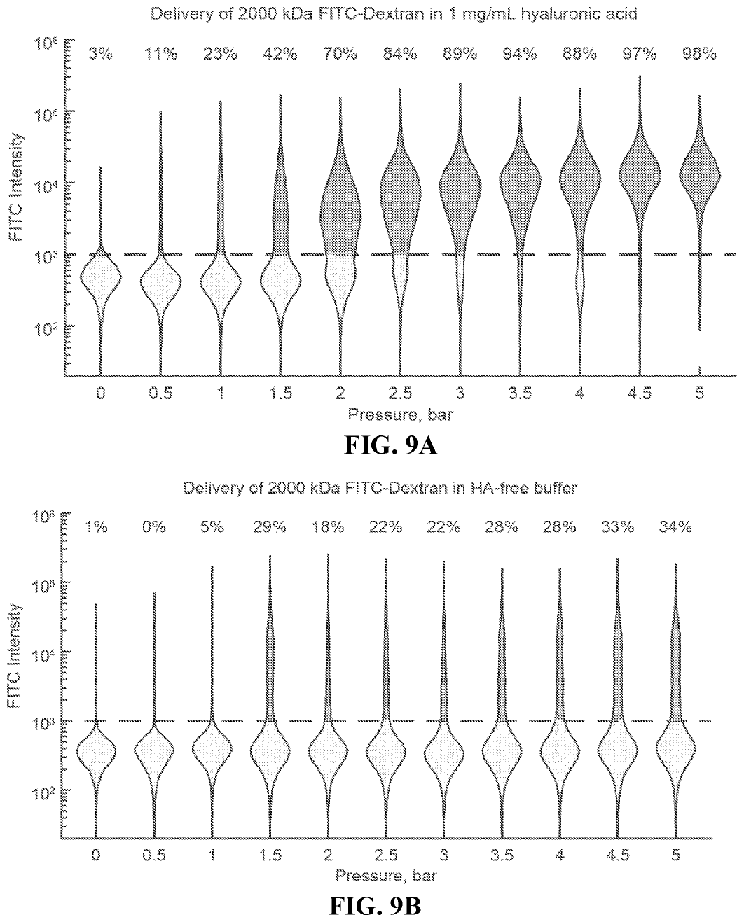

A high-speed camera and brightfield microscopy was used to quantify cell deformation in microfluidic extensional flow. Cells were suspended in a solution of about 0.5% poly(ethylene oxide) and pumped at three difference flow rates: 0.1 ml/min, 0.32 ml/min, and 1 ml/min (FIG. 7A). A convolutional neural network (U-Net) was trained in MATLAB to automatically segment cells and measure deformation. (FIG. 7B). Altogether, this confirmed that viscoelastic extensional flow resulted in significant cell elongation. FIGS. 8A and 8B show quantification of cell deformation in viscoelastic extensional flow with high-speed microscopy and automated image segmentation at 0.05 bar and 2.5 bar respectfully. Together, these results show cell deformations are large and deformation of the cell to an aspect ratio of at least 2 may be required for membrane permeabilization.

To optimize intracellular delivery of large cargos, microfluidic micro-channels were fabricated using PDMS glass microfabrication. Jurkat cells were suspended in phosphate buffer at a concentration of up to about 1×10⁸ cells/ml with 2,000 kDa FITC-Dextran at a concentration of about 0.2 mg/mL and 1,500 kDa HA at concentrations of up to about 2 mg/mL, resulting in a highly viscoelastic solution. 2,000 kDa dextran is about 25 nm in size (much larger than, e.g., a Cas9/sgRNA ribonucleoprotein complex (RNP), which is about 13 nm in size) and a benchmark standard for intracellular delivery. For a typical condition, 40 μL of cell suspension containing about 1 million cells were pumped through the micro-channel at a regulated pressures of up to about 5 bar. Cell stretching was performed at room temperature and took about 0.5 to 2 seconds per sample. ‘Zero flow rate’ samples were incubated with the delivery solution without stretching. After stretching, cells were immediately diluted in prewarmed culture medium, washed, and cultured overnight. Cell viability (propidium iodide exclusion) and FITC-dextran delivery were assessed by imaging flow cytometry (Amnis ImageStream) the next day.

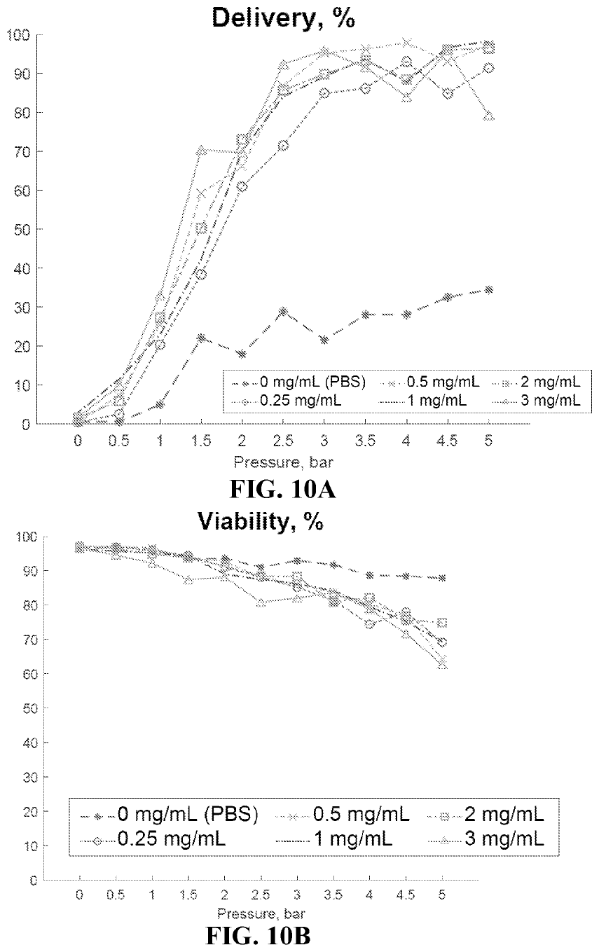

As shown in FIG. 9A, efficient delivery to over about 90% of viable cells was observed when using a delivery suspension containing about 1 mg/mL HA at pressures above 3 bar. Contrastingly, as shown in FIG. 9B, the delivery efficiency did not exceed 35% when using HA-free PBS (i.e., the Newtonian delivery suspension).

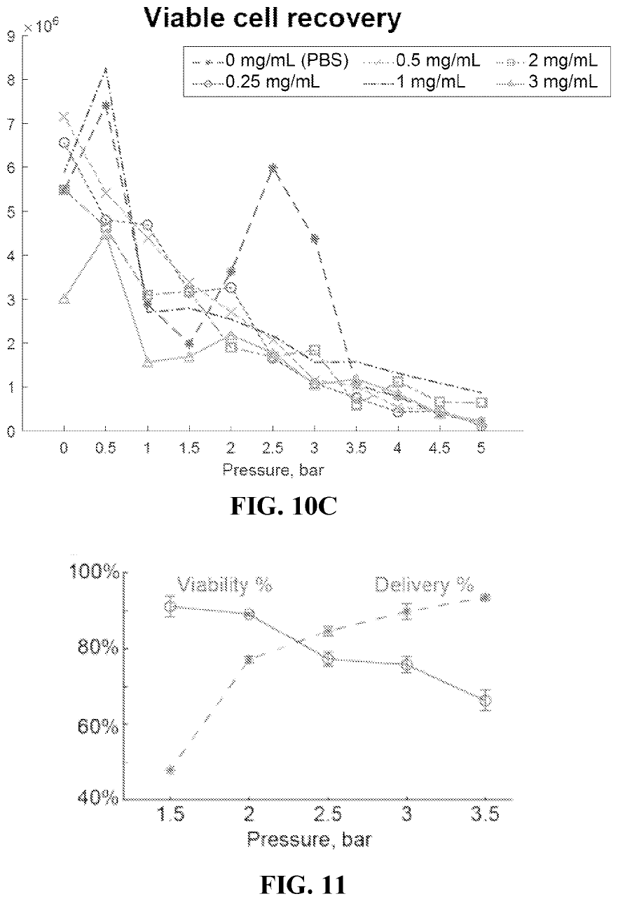

Furthermore, a range of HA concentrations was tested, and it was observed that the delivery efficiency, viability, and total cell yield as a function of driving pressure were similar for HA concentrations between about 0.5 mg/mL and about 3 mg/mL (FIGS. 10A-10C). At HA concentrations above 3 mg/mL, the solution becomes highly viscous and strongly shear thinning due to the entanglement between HA filaments. Additionally, cell viability and yield were reduced at HA concentrations above 3 mg/ml.

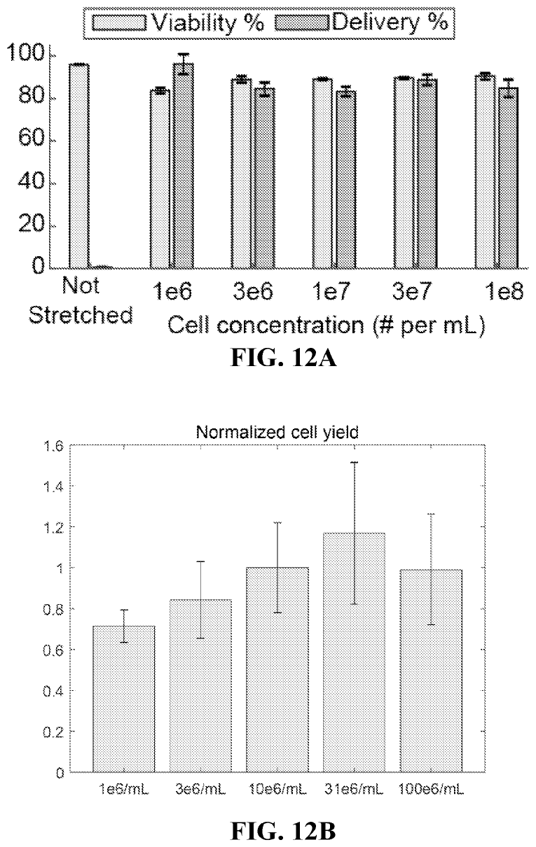

As expected, delivery efficiency of 2,000 kDa FITC-dextran increased with increasing device pressure (FIG. 11). Fluorescence microscopy imaging confirmed diffuse delivery of FITC-dextran to the cytosol. At 3 bar, the flow rate through a single micro-channel was about 3 ml/min.

To investigate the effect of cell concentration, cell concentrations of up to 1×10⁸ cells per ml in the delivery suspension were tested. No substantial effect was observed on the delivery efficiency of 2,000 kDa FITC-dextran, cell viability, or cell yield at higher cell concentrations (FIGS. 12A-12B). Thus, the results show the ability to use very high cell densities (up to 1e8 cells/ml) with the methods and systems of the disclosure. This is yet another unique and advantageous property of the methods and systems disclosed herein given that other emerging mechanoporation strategies require cells to be dilute (e.g., at about 5×10⁶ cells/ml or even lower than about 5×10⁶ cells/ml) and therefore, require much larger amounts of expensive cargo in the cell suspension in order to achieve significant intracellular delivery to each cell.

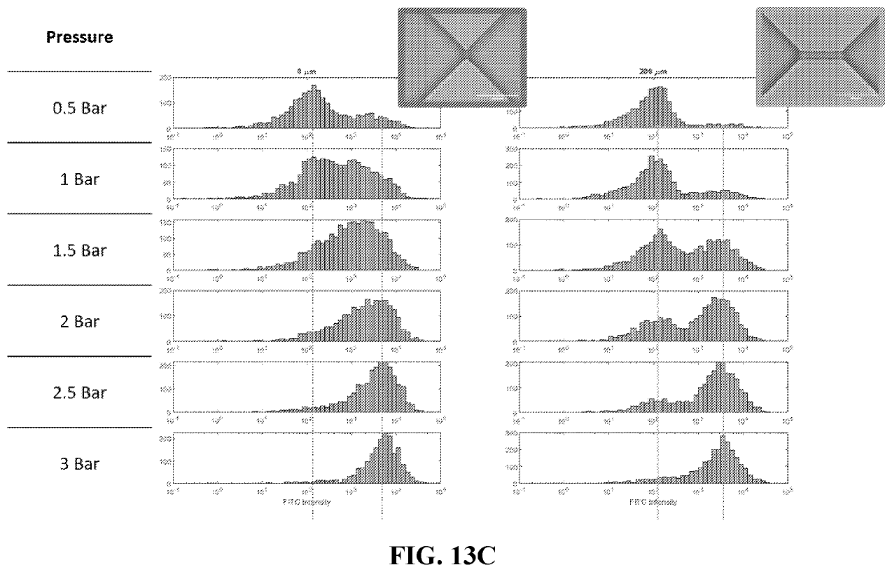

The effect of channel geometry on delivery efficiency was also investigated by using micro-channels having a length of 0 μm and 200 μm. It was found that a “zero-length channel” (e.g., a micro-channel having a length of about 0 μm or a pore) still resulted in efficient delivery of 2,000 kDa FITC-dextran at higher flow rates, however, at the expense of cell viability (FIGS. 13A and 13B). Furthermore, as shown in FIG. 13C, the existence of a long channel (e.g., at least 100 μm in length) resulted in a clear bimodal distribution in FITC-dextran intensity, suggesting efficient delivery to all permeabilized cells.

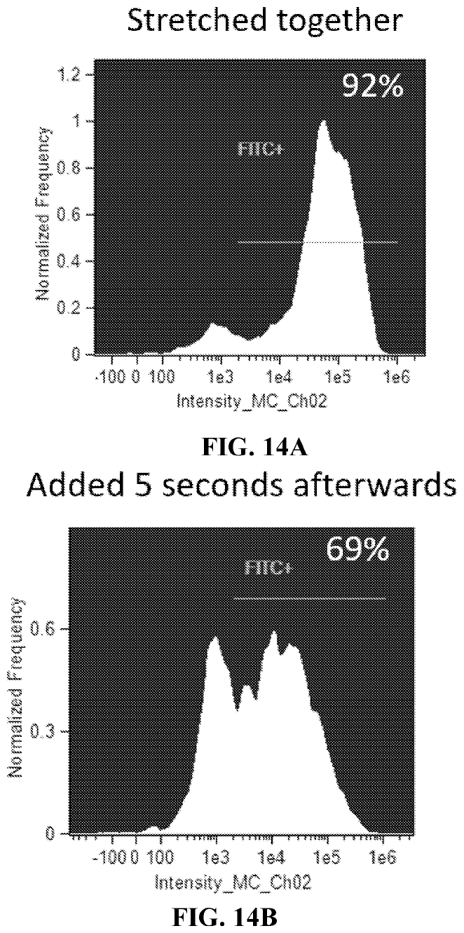

To learn more about the mechanism and timescale of intracellular delivery, cells were first stretched and then 2,000 kDa FITC-dextran was added to the delivery suspension within 5 seconds after stretching the cells. The delivery efficiency and amount of delivered dextran were both greatly reduced (FIGS. 14A-14C). The effect of dilution timing on delivery efficiency or delivery amount was also investigated. An experiment was further performed where cells were stretched with the 2,000 kDa FITC-dextran cargo included in the delivery suspension during stretching (as is typical), and cells were immediately diluted 10-fold in culture medium, versus waiting for up to five minutes after stretching to dilute the cells (FIG. 14D). There was no difference observed across all conditions. Together these data suggest that the delivery process is rapid, on the timescale of seconds, since adding the cargo molecule after a few seconds results in substantially reduced delivery, and conversely, diluting away the cargo in the delivery suspension even just a few seconds after cell stretching had no discernable effect on the delivery efficiency or amount.

### Example 3. Intracellular Delivery to Primary T Cells and Acute Myeloid Leukemia Cells

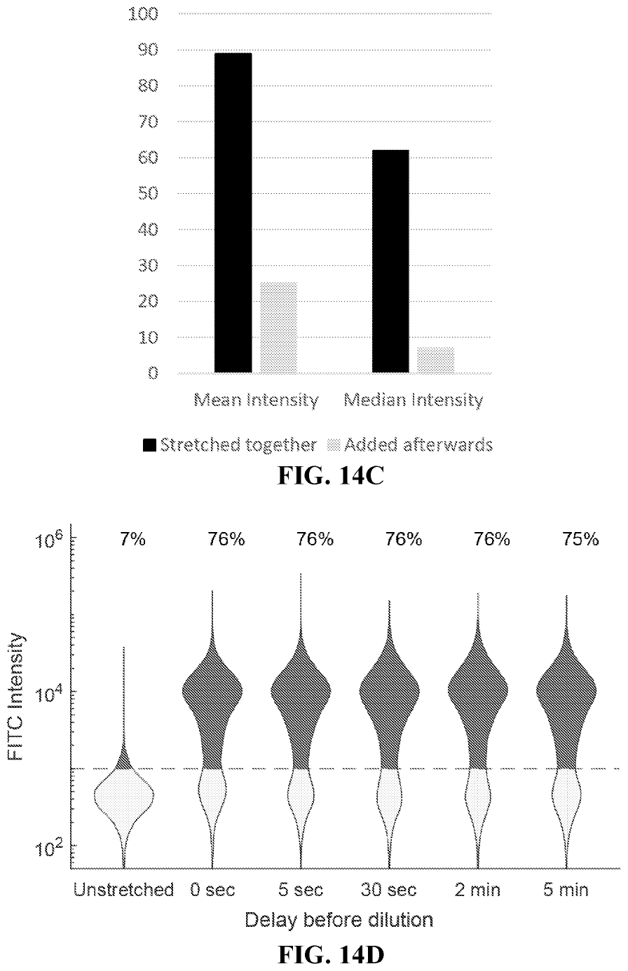

Primary CD3+ T cells were isolated and expanded from whole blood from healthy donors by CD3/CD28 magnetic bead positive isolation after IL-2 stimulation. CD3, CD4, and CD8 were quantified by intracellular cytokine staining (ICC) after three days. Over 95% of cells were found to be CD3+, and the CD4/CD8 ratio was about 2.4. Five days after isolation, 2,000 kDa FITC-Dextran was delivered to the expanded primary CD3+ T cells by cell stretching using the methods and systems of the disclosure. The primary CD3+ T cells were washed and returned to culture conditions, and the delivery of 2,000 kDa FITC-Dextran was assessed the next day as a function of pressure (FIG. 15). Up to about 80% delivery efficiency was observed at pressures higher than about 3 bar. These results demonstrate the feasibility of delivery of large cargoes (e.g., about 25 nm in size and larger than RNP) to primary T cells at over 100 million cells per minute using a single microchannel of the disclosure.

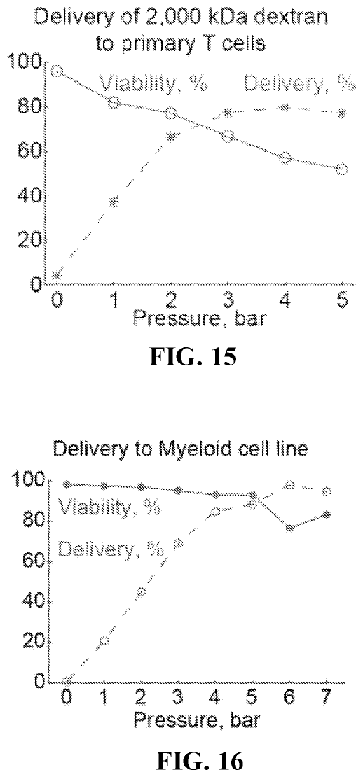

The delivery of 70 kDa FITC-Dextran to MOLM-13 cells, an acute myeloid leukemia cell line, was evaluated. MOLM-13 cells are known to be challenging to successfully electroporate. Over about 90% delivery efficiency of 70 kDa FITC-Dextran in MOLM-13 cells was observed with no further optimization (FIG. 16). Up to about 90% delivery efficiency was observed at pressures higher than about 4 bar. These results support the feasibility of applying this technique to multiple cell types and show that viscoelastic cell stretching enables efficient delivery to other cell types that are, for example, known to be difficult to transfect, such as acute myeloid leukemia cells.

### Example 4—Viscoelastic Cell Stretching for Intracellular Delivery of RNP to Jurkat Cells

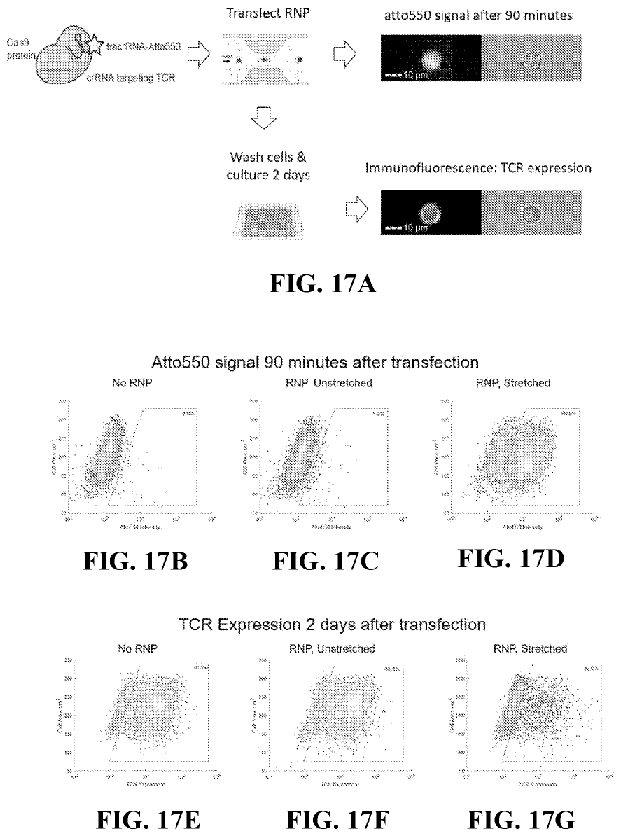

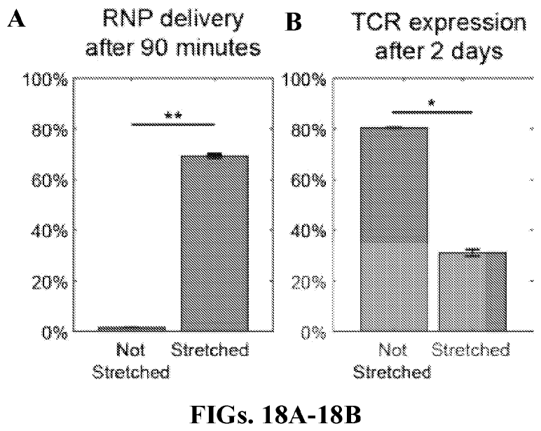

Next, RNP delivery to Jurkat cells was tested. Cas9 protein was complexed with: 1) a CRISPR RNA (crRNA), which targets the alpha constant locus of the T cell receptor and 2) trans-activating crRNA (tracrRNA) conjugated to the fluorophore ATTO™ 550. The fluorescently labeled crRNA:tracrRNA Ribonucleoprotein Complex (RNP) complex was then delivered to Jurkat cells at 3 bar using cell stretching methods and systems of the disclosure (FIG. 17A). Cells were washed and RNP transfection efficiency was measured 90 minutes after transfection in negative controls (i.e., no RNP present in cell suspension; FIG. 17B), unstretched cells contacted with the cell suspension containing RNP (FIG. 17C), and stretched cells contacted with the cell suspension containing RNP (FIG. 17D). As with the FITC-dextran experiments, “unstretched” cells were incubated with RNP and delivery suspension but not processed through the micro-channel. RNP transfection efficiency was found to be about 70% in stretched cells exposed to RNP (FIG. 18A).

Cells were cultured for 2 days and T cell receptor (TCR) expression was quantified by ICC in negative controls (i.e., no RNP present in cell suspension; FIG. 17E), unstretched cells contacted with the cell suspension containing RNP (FIG. 17F), and stretched cells contacted with the cell suspension containing RNP (FIG. 17G). TCR expression was found to be about 80% for both untreated (FIG. 17E), and unstretched cells (FIGS. 17F and 18A) and about 31% for the stretched cells (FIG. 17G and FIG. 18B), thereby indicating a knockout efficiency of about 61%. Cell viability two days after RNP transfection was about 94% versus 97% in the controls. Altogether, these results demonstrate efficient delivery of a large biomolecule (e.g., about 13 nm in size) to T cells and establish the feasibility of very fast (e.g., within about 5 seconds or less) RNP transfection and gene editing of T cells.

### Example 5. Viscoelastic Cell Stretching for RNP Intracellular Delivery to T Cells

The viscoelastic cell stretching methods disclosed herein are developed into a reliable method for delivering proteins into cells at one billion cells per minute. High throughput T cell transfection with RNP is tested because of the urgent need for billions of T cell knockouts for allogenic CAR T cell therapies.

### Optimization of Microfluidic System for Ultra-High Throughput Intracellular Delivery

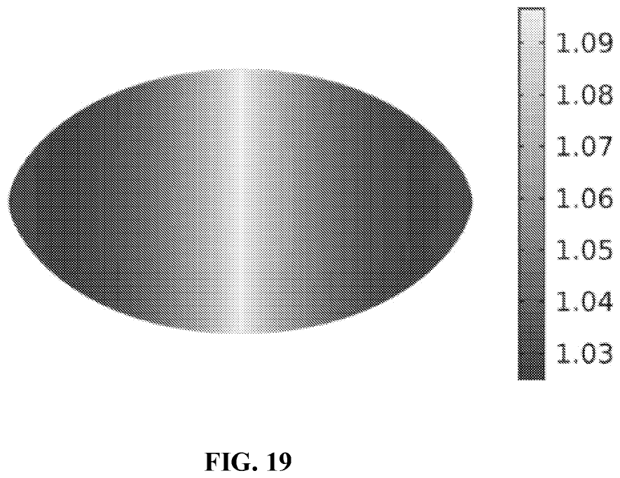

The microfluidic geometry and polymer concentration and composition are optimized to maximize delivery and cell viability. Delivery of 4 kDa-2,000 kDa FITC-Dextran to Jurkat cells is evaluated by flow cytometry. The effect of increasing the channel constriction ratio (e.g., the ratio of a hydraulic diameter of the chamber to a hydraulic diameter of the micro-channel), which increases both extensional stresses and strains, is investigated. Experimental results are corroborated with computational fluid dynamics simulations of viscoelastic flow in OpenFoam and RheoToo167 and finite element simulations of cell deformation in response to flow forces (e.g., similar to the simulations presented in FIG. 19).

### Effects of Cell Stretching on T Cells

Several key metrics of cellular damage at multiple timepoints after stretching are assayed by ICC, namely mitochondrial membrane potential (MitoTracker™), apoptosis/membrane composition (Annexin V staining), oxidative stress (CellROX™) and double-stranded DNA breaks (immunocytochemical detection of phosphorylated histone H2A.X at position Ser₁₃₉). Phenotypic changes due to cell stretching and permeabilization are assessed by assaying T cell activation (expression of CD69) and exhaustion (expression of programmed cell death protein-1 (PD-1)).

### Optimization of Viscoelastic Cell Stretching for RNP-Mediated Knockout of T Cell Receptor

The viscoelastic cell stretching for RNP delivery to Jurkat cells is optimized, building on preliminary results (e.g., FIGS. 18A and 18B). TCR is knocked out from Jurkat cells by cleavage of the TCR alpha constant gene (TRAC). Previously-published sgRNA sequences shown to target TRAC with high specificity are used. RNPs are formed in vitro by mixing commercial recombinant S. pyogenes Cas9 protein with synthetic crRNA and tracrRNA or single guide RNA and then transfected by cell stretching. As assessed in previous experiments described above, direct delivery of RNP is measured by fluorescently-tagged RNP and TCR knockout is evaluated after 48 hours by immunocytochemistry. On-target and off-target DNA cutting is assessed by deep sequencing of several loci for this specific crRNA.

### Integration of Inertio-Elastic Focusing and Scale Up to One Billion Cells Per Minute

To improve uniformity of the cell stretching process, inertio-elastic cell focusing is implemented into the microfluidic design. Positioning all cells to the same lateral position ensures that the same stretching force is applied to every cell for the same length of time. Very fast cell focusing along a flow path line in viscoelastic solutions is performed at flow rates of up to 20 mL/min. A multi-, micro-channel device (e.g., a device including two or more micro-channels of the disclosure) capable of processing one billion cells per minute is developed and used. This work yields an ultra-high throughput technique for efficient ex vivo delivery of DNA-editing proteins and characterization of key mechanisms of cell damage.

### Example 6. Ultra-Fast Production of Allogenic Chimeric Antigen Receptor (CAR) T Cells

Viscoelastic cell stretching is used to efficiently produce TCR-negative CAR-T cells with CRISPR at one billion cells per minute. RNP transfection to fragile primary T cells is optimized. Then, CRISPR RNPs is delivered alongside CAR transgenes, specifically a construct developed previously for treating gliomas targeting the EGFRvIII mutation is used.

High Throughput CRISPR Knockout of TRAC from Primary T Cells by Cell Stretching

CD3+ Pan T cells are isolated from whole blood from healthy donors by magnetic negative selection, then activated and expanded by stimulation with CD3/CD28 beads and IL-2 for about 3-6 days before delivery of fluorescent dextran or RNP targeting TRAC. As described above, markers of mitochondrial integrity, DNA damage, and oxidative stress are assessed at multiple timepoints following cell stretching. Delivery efficiency, viability, proliferation, CD4/CD8 ratio, activation (CD69 expression), exhaustion (PD-1 expression), TCR knockout, and off-target DNA cutting are evaluated by the immunocytochemistry and deep sequencing as described above.

### Production of CAR-T Cells

The primary TRAC-knockouts optimized as described above are augmented with AAV6 transduction of the EGFRvIII CAR transgene onto the endogenous TRAC promoter. The transgene is packaged and delivered by adeno-associated viral vector AAV6, as is common for CAR-T generation as naked cytosolic DNA can be stressful to T cells. Although cytosolic dsDNA is generally expected to be toxic, unexpectedly low toxicity in primary T cells has been observed when delivered with RNP. Direct delivery of dsDNA templates is also evaluated. CAR integration and expression is confirmed by deep sequencing of the TRAC locus and ICC.

### In Vitro Functional Studies of CAR T Cells

High throughput in vitro assays of tumor cell killing using xCELLigence® real-time cell analysis system is performed. This analysis system provides impedance-based measurements of both CAR-T cell and tumor cell densities in a microwell plate format during co-incubation. CAR-T cells are seeded with U87vIII, a glioma cell line which expresses the EGFRvIII mutation, and cell densities of both CAR-T and U87vIII are tracked dynamically for up to five days.

The methods are performed with minimizing damages and changes to primary T cells. Potential effects of intracellular calcium, ATP depletion, and oxidative stress can be minimized by the addition of antioxidants (e.g. Trolox, a vitamin E analog), poloxamer (e.g., PluronicR® F-68, a membrane-stabilizing amphiphile that helps seal membrane pores71), apoptosis inhibitors (e.g., Q-VD-Oph, a small molecule pan-Caspase inhibitor), glucose, and other supplements commonly used in cryo recovery media to the cell suspension.

This work yields an optimized protocol and in vitro validation of allogenic CAR-T cells produced from primary cells.

### Example 7. High Throughput Cell “Surgery” for Protein Delivery and Precision Gene Editing

Research applications frequently require quickly transfecting many samples as part of the same experiment, either to optimize delivery conditions, screen a wide range of genomic perturbations, or generate libraries of differently modified cells to allow selection of a favorable phenotype. An automated system that is compatible with microwell plates is developed. The automated system allows integration with high-throughput workflows & instruments.

As described above, a major challenge in precision gene editing is the inability to direct the process of DNA repair towards the desired outcome. There is strong evidence that the frequency of homology directed repair (HDR) can be greatly increased if DNA repair factors such as Rad52 are delivered to the cytosol. Direct protein delivery is expected to bypass the need for inducer-controlled expression because endogenous proteases normally degrade most proteins within 24 hours. Intracellular delivery of Rad52 protein may enable more complete HDR-mediated gene editing during the crucial window of time following formation of the specific double-stranded DNA break, while also safely avoiding any long-term alteration to the homeostatic equilibrium between competing DNA repair pathways.

### High Throughput Cell ‘Surgery’ in Microwell Plates

An automated device for viscoelastic cell stretching that is compatible with microwell plates was developed. The geometries of the micro-channels of the disclosure, described above, were fabricated by stereolithography three-dimensional (3D) printing, which has an isotropic tolerance of about 25 μm. FIGS. 6A-6C show cross-sectional, side/perspective, and top/perspective views, respectively, of an example three-dimensional printed, microfluidic device 600 compatible with microwell plates. The microfluidic device 600 has been described in more detail elsewhere herein. A 3-axis positioning system, a liquid handling system, and a probe rinsing station are integrated into the microfluidic system to allow reproducible and automatic processing of multiple samples.

### Delivery of Rad52 Protein to Enhance Gene Editing Efficiency

Recombinant Rad52 is produced in E. coli using T7 expression and pET vector with an N-terminus polyhistidine tag for purification, following existing protocols. At 46 kDa, Rad52 is small so efficient intracellular delivery using the methods and systems of the disclosure, after routine optimization, is expected. HDR efficiency is assessed in a previously established model system which uses HEK293 cells with a broken green fluorescent protein (GFP) cassette as well as an optimized guide RNA and single-stranded oligonucleotide donor template. This model system has a baseline repair HDR frequency of about 15% that is increased several-fold by Rad52. Furthermore, validation of the delivery of Rad52 is performed and intracellular half-life and localization is measured by fluorescent labeling of the protein and in situ immunostaining.

Alternative 3D microfabrication technologies such as micro-machining, EDM, injection molding, and nanolithography may also be used. If necessary, localization of recombinant Rad52 to the nucleus is enhanced by adding a C-terminus nuclear localization sequence.

This work results in the first automated system for protein transfection for diverse cell types, as well as the first evidence of DNA repair factors delivered intracellularly in protein form to safely enhance HDR.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-49. (canceled)

50. A method of intracellular delivery of a substance to one or more cells, the method comprising:
providing a device comprising a substrate defining a micro-channel in fluid communication with a first chamber and optionally in fluid communication with a second chamber, the micro-channel having a hydraulic diameter that is less than a hydraulic diameter of the first and second chambers; and
driving a cell suspension through the micro-channel, thereby: i) causing the one or more cells to be stretched along a direction of flow and ii) inducing a formation of one or more temporary pores in a membrane of the one or more cells,
wherein the cell suspension comprises the one or more cells, a polymer, and the sub stance.

51. The method of claim 50, wherein the substance comprises a protein, a nucleic acid, a nanomaterial, a drug, a catalyst, a polysaccharide, a bacterium, an organelle, a contrast agent, a molecular probe, a dye, or any combination thereof.

52. The method of claim 50, wherein the one or more cells are maximally stretched at a circumferential region around or near a center of each of the one or more cells, and wherein the driving of the cell suspension through the micro-channel causes an advection of the cell suspension toward the circumferential region.

53. The method of claim 52, wherein the driving of the cell suspension through the micro-channel creates a viscoelastic extensional flow that is a uniaxial extensional flow, a biaxial extensional flow, or a combination thereof.

54. The method of claim 50, wherein the cell suspension is driven through the micro-channel at a volumetric flow rate ranging from about 0.1 ml/minute to about 20 ml/minute or at a volumetric flow rate resulting in a Weissenberg number ranging from about 1 to about 100.

55. The method of claim 50, wherein the cell suspension is driven through the micro-channel by applying a driving pressure ranging from about 20 millibar (mbar) to about 7 bar.

56. The method of claim 50, wherein the cell suspension has one or more viscoelastic properties, and wherein the cell suspension is not a Newtonian fluid.

57. The method of claim 56, wherein the one or more temporary pores have a diameter ranging from about 5 nm to about 100 nm, and wherein the one or more temporary pores are sealed by the one or more cells within about 20 seconds after their formation.

58. The method of claim 50, wherein the polymer comprises hyaluronic acid, poly(ethylene) glycol, poly(vinyl) pyrrolidone, poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactides (PLA), polyglycolides (PGA), poly(lactide-co-glycolides) (PLGA), polyanhydrides, polyorthoesters, polycyanoacrylate polycaprolactone, cellulose, lignin, alginate, chitosan, starch, or any combination thereof.

59. The method of claim 50, further comprising focusing the one or more cells into a localized fluid flow path, wherein the one or more cells are located within a same distance from each other.

60. A method of delivery of a substance to one or more particles, the method comprising:
providing a substrate defining a micro-channel that connects a first chamber and a second chamber, the micro-channel having a hydraulic diameter that is less than a hydraulic diameter of the first and second chambers; and
driving a particle suspension through the micro-channel, thereby: i) causing the one or more particles to be stretched along a direction of flow and ii) inducing a formation of one or more temporary pores in a membrane of the one or more particles,
wherein the particle suspension comprises the one or more particles, a polymer, and the substance.

61. A microfluidic system for intracellular delivery of a substance to one or more cells, the microfluidic system comprising:
a substrate defining a micro-channel that connects a first chamber and optionally a second chamber, the micro-channel having a hydraulic diameter that is less than a hydraulic diameter of the first and second chambers;
a cell suspension comprising the one or more cells, a polymer, and the substance;
a pump configured to drive the cell suspension through the micro-channel, thereby: i) causing the one or more cells to be stretched along a direction of flow and ii) inducing a formation of one or more temporary pores in a membrane of the one or more cells; and
optionally comprising a controller configured to control operation of the pump.

62. The microfluidic system of claim 61, wherein the hydraulic diameter of the micro-channel is greater than a diameter of the one or more cells.

63. The microfluidic system of claim 62, wherein the diameter of the one or more cells ranges from about 4 μm to about 25 μm, wherein the hydraulic diameter of the micro-channel ranges from about 30 μm to about 100 μm, and wherein the hydraulic diameter of the first and second chambers ranges from about 1,000 μm to about 10,000 μm.

64. The microfluidic system of claim 61, wherein the first and second chambers are tapered such that the hydraulic diameter of the first and second chambers gradually narrows prior to intersecting an opening of the micro-channel.

65. The microfluidic system of claim 64, wherein a ratio of the cross-sectional area of the first or second chambers to the cross-sectional area of the micro-channel ranges from about 10 to about 500.

66. The microfluidic system of claim 61, wherein the pump is configured to drive the cell suspension from the first chamber, through the micro-channel, and to the second chamber.

67. The microfluidic system of claim 61, wherein the controller is configured to control the pump to drive the cell suspension through the micro-channel at a volumetric flow rate ranging from about 0.1 ml/minute to about 20 ml/minute or at a volumetric flow rate resulting in a maximum Reynolds number of a flow ranging from about 10 to about 100.

68. The microfluidic system of claim 61, wherein the micro-channel is a first micro-channel and the microfluidic system further comprises a second or more micro-channels connecting the first or second chambers to a third or more chambers in parallel.

69. The microfluidic system of claim 61, further comprising a valve in fluid communication with the micro-channel and a pressure line, the valve configured to selectively control a driving pressure applied by the pump.
